(19) United States
(12) Patent Application Publication
(10) Pub. No.: US 2024/0182413 A1
(43) Pub. Date: Jun. 6, 2024

(54) INSECTICIDAL COMPOUNDS

(71) Applicant: Pluton Biosciences, Inc., St. Louis, MO (US)

(72) Inventors: Farhan James William, St. Louis, MO (US); Timothy Adam Wencewicz, St. Louis, MO (US); Ann Marie Guggisberg, Richmond Heights, MO (US)

(21) Appl. No.: 18/388,444

(22) Filed: Nov. 9, 2023

Related U.S. Application Data
(60) Provisional application No. 63/424,935, filed on Nov. 13, 2022.

(51) Int. Cl. C07D 209/08; A01N 43/38; A01N 43/40; A01P 7/04; C07D 213/22

(52) U.S. Cl. CPC C07D 209/08; A01N 43/38; A01N 43/40; A01P 7/04; C07D 213/22

(57) ABSTRACT

Provided herein is technology relating to particular heterocyclic compounds, including compounds that comprise structural modifications of arundine and substituted derivatives of arundine, and particularly, but not exclusively, to use of arundine analogs as an insecticide, methods of making arundine analogs, and methods of using arundine analogs for killing insects.

### STATEMENT OF RELATED APPLICATIONS

This application claims priority to U.S. Provisional Patent Application No. 63/424,935, filed Nov. 13, 2022, the entire contents of which are incorporated herein by reference for all purposes.

### FIELD

Provided herein is technology relating to particular heterocyclic compounds, including compounds that comprise structural modifications of arundine and substituted derivatives of arundine, and particularly, but not exclusively, to use of arundine analogs as an insecticide, methods of making arundine analogs, and methods of using arundine analogs for killing insects.

### BACKGROUND

Insect pests cause significant loss of agricultural yields and are vectors for disease transmission in plants, animals, and humans. Effective insecticides provide one strategy for reducing insect-associated problems, but insecticide efficacy is continually threatened by resistance. New chemical scaffolds and modes of action are needed to combat insect pests and to address the associated threats to agricultural yield and human health. See, e.g., Sparks (2015) “IRAC: Mode of Action Classification and Insecticide Resistance Management” Pesticide Biochemistry and Physiology 121: 122-128.

### SUMMARY

Embodiments of the present technology relate generally to insecticidal compositions and associated methods that are effective against insects. Previous research has shown that 3,3-diindolylmethane (arundine) and 3-((1H-indol-2-yl)methyl)-1H-indole) have insecticidal activity against some insects. See, e.g., U.S. patent application Ser. No. 17/724,078, which is incorporated by reference herein in its entirety. For example, the scaffold compound 3,3-diindolylmethane caused greater than 95% mortality for the lepidopteran fall armyworm (Spodoptera frugiperda) after seven days of exposure of armyworms to the scaffold compound. See, e.g., U.S. patent application Ser. No. 17/724,078, especially at Tables 3 and 4 therein.

Accordingly, the technology provided herein provides improved insecticidal compounds (e.g., particular heterocyclic compounds disclosed herein such as certain arundine analogs (e.g., bisindole compounds)), methods of making improved insecticidal compounds, and methods of killing insects using insecticidal compounds. In some embodiments, the technology provided herein relates to a bisindolylmethane scaffold that finds use in the design and synthesis of insecticidal compounds. For example, the technology provided herein provides specific arundine analogs (e.g., bisindole compounds) that are derivatives of arundine or 3-((1H-indol-2-yl)methyl)-1H-indole.

Data collected during the development of embodiments of the technology provided herein indicated that some specific bisindole compounds have an increased insecticidal activity with respect to arundine and 3-((1H-indol-2-yl)methyl)-1H-indole. In addition, data collected during the development of embodiments of the technology disclosed herein indicated that bisindole compounds have increased insecticidal activity relative to monoindole compounds and, further, that bisindole compounds have increased insecticidal activity relative to compounds comprising more than two indolyl moieties (e.g., trisindole and tetraindole compounds). Furthermore, data collected during the development of embodiments of the technology described herein indicated that bisindole compounds comprising at least two bonds (e.g., at least two carbon-carbon bonds) in the linker connecting the two indolyl moieties (e.g., having a general structure of indolyl-C-indolyl) have increased insecticidal activity relative to bisindole compounds comprising one bond (e.g., one carbon-carbon bond) connecting the two indolyl moieties (e.g., indolyl-indolyl). That is, bisindole compounds comprising at least one atom (e.g., one carbon atom) in the linker connecting the two indolyl moieties have increased insecticidal activity relative to bisindole compounds comprising no atoms in the linker connecting the two indolyl moieties. Data collected during the development of embodiments of the technology described herein indicated that substituents may be added to the linker connecting the two indolyl moieties without substantially changing the insecticidal activity of the bisindole compounds. Moreover, data collected during the development of embodiments of the technology described herein indicated that some specific substituents added to the bisindole linker increased the insecticidal activity of the bisindole compound relative to the insecticidal activity of the bisindole compound without the substituent added to the bisindole linker. Finally, data collected during the development of embodiments of the technology described herein indicated that bisindole compounds comprising certain substituents on the indolyl rings (e.g., a halogen substituent (e.g., F, I, Cl, or Br)) had an increased insecticidal activity relative to compounds that did not comprise the modifications of the indolyl rings.

Accordingly, provided herein is technology related to a compound comprising a structure according to:

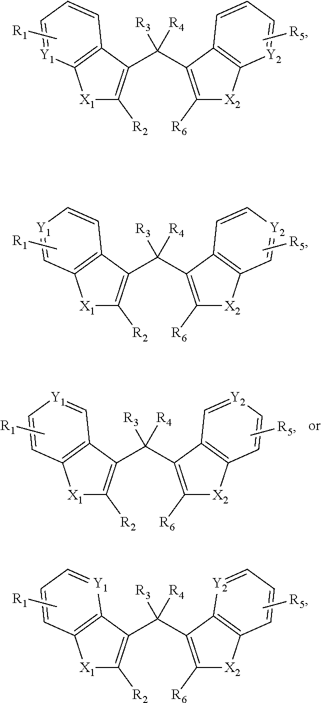

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl. In some embodiments, R₃ or R₄ comprises an aryl. In some embodiments, R₃ or R₄ comprises an aryloxy. In some embodiments, R₃ or R₄ comprises a phenoxy. In some embodiments, R₁ or R₅ comprises a halogen. In some embodiments, R₁ or R₅ comprises a fluorine. In some embodiments, X₁ or X₂ is N. In some embodiments, Y₁ or Y₂ is C.

In some embodiments, the technology is related to a compound comprising a structure according to:

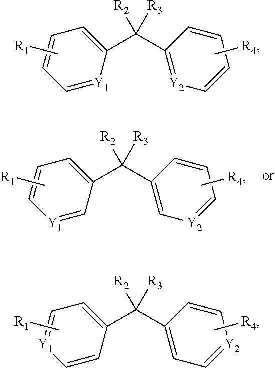

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂. In some embodiments, R₂ or R₃ comprises an aryl. In some embodiments, R₂ or R₃ comprises an aryloxy. In some embodiments, R₂ or R₃ comprises a phenoxy. In some embodiments, R₁ or R₄ comprises a halogen. In some embodiments, R₁ or R₄ comprises a fluorine. In some embodiments, Y₁ or Y₂ is N.

In some embodiments, the technology relates to a compound comprising a structure according to:

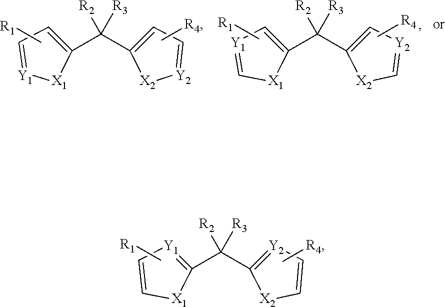

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, or S, wherein R₅ is H, alkyl, or aryl; and X₂ is CH—R₆, N—R₆, O, or S, wherein R₆ is H, alkyl, or aryl. In some embodiments, R₂ or R₃ comprises an aryl. In some embodiments, R₂ or R₃ comprises an aryloxy. In some embodiments, R₂ or R₃ comprises a phenoxy. In some embodiments, R₁ or R₄ comprises a halogen. In some embodiments, R₁ or R₄ comprises a fluorine. In some embodiments, X₁ or X₂ is N. In some embodiments, Y₁ or Y₂ is C.

In some embodiments, the technology provides a method of synthesizing an insecticidal compound. For example, in some embodiments, methods comprise reacting a heterocyclic molecule and a ketone or aldehyde. In some embodiments, the heterocyclic molecule comprises an indole. In some embodiments, the insecticidal compound is an arundine analog. In some embodiments, the insecticidal compound is a bisindole compound.

In some embodiments, the insecticidal compound comprises a structure according to:

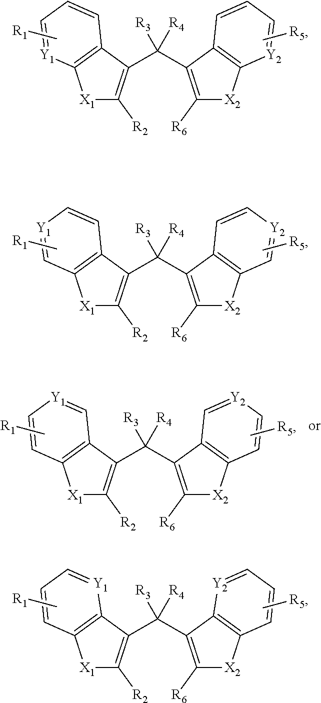

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl.

In some embodiments, the insecticidal compound comprises a structure according to:

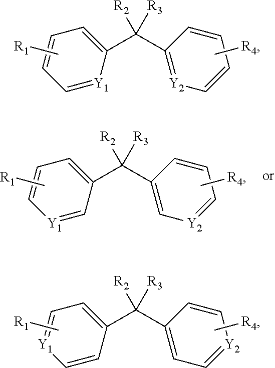

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂.

In some embodiments, the insecticidal compound comprises a structure according to:

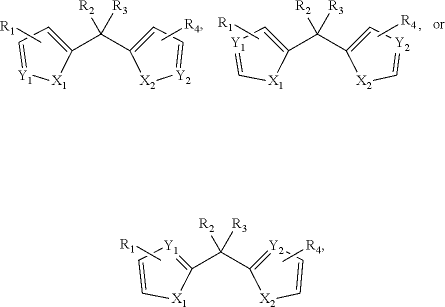

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, or S, wherein R₅ is H, alkyl, or aryl; and X₂ is CH—R₆, N—R₆, O, or S, wherein R₆ is H, alkyl, or aryl.

In some embodiments, the technology provides a method of killing an insect. For example, in some embodiments, methods of killing an insect comprise contacting an insect with a compound having a structure according to:

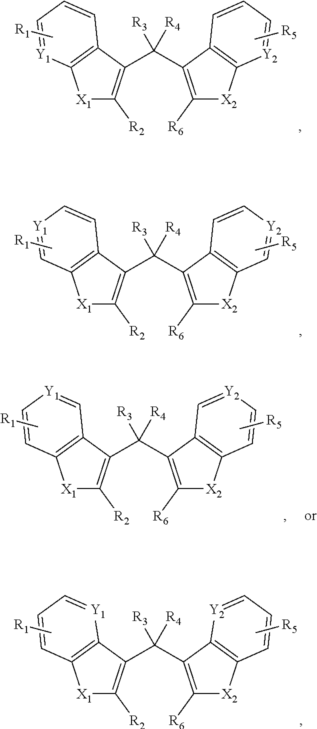

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl.

In some embodiments, methods of killing an insect comprise contacting an insect with a compound having a structure according to:

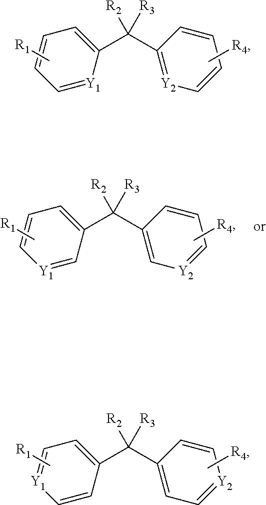

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂.

In some embodiments, methods of killing an insect comprise contacting an insect with a compound having a structure according to:

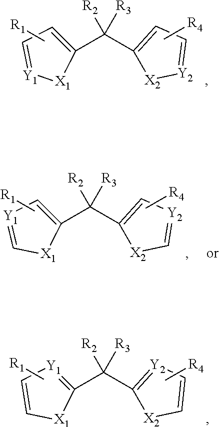

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, or S, wherein R₅ is H, alkyl, or aryl; and X₂ is CH—R₆, N—R₆, O, or S, wherein R₆ is H, alkyl, or aryl.

In some embodiments, the compound is provided at a concentration of less than 30 micromolar. In some embodiments, the compound is provided at a concentration of less than 20 micromolar. In some embodiments, the compound is provided at a concentration of less than 10 micromolar. In some embodiments, the compound is provided at a concentration of less than 1 micromolar. In some embodiments, the compound is provided at a concentration of less than 100 nanomolar. In some embodiments, the compound is provided at a concentration of less than 10 nanomolar. In some embodiments, the compound is provided at a concentration of less than 1 nanomolar.

The technology is not limited in the mode of the compound contacting an insect. For example, in some embodiments, contacting an insect comprises the insect ingesting the compound, the compound contacting a cuticle or exoskeleton of the insect, or the compound passing through a spiracle of the insect.

In some embodiments, the technology provides an insecticidal composition comprising a compound having a structure according to:

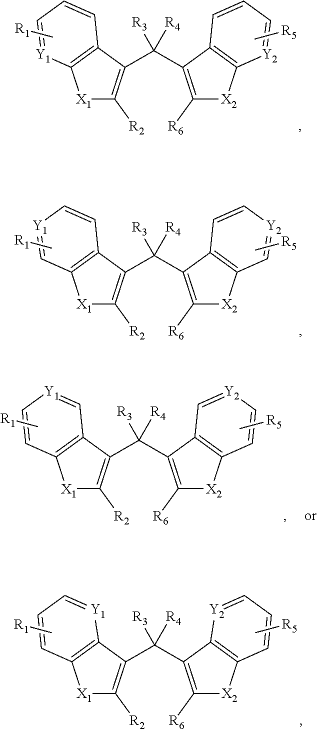

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl.

In some embodiments, the technology provides an insecticidal composition comprising a compound having a structure according to:

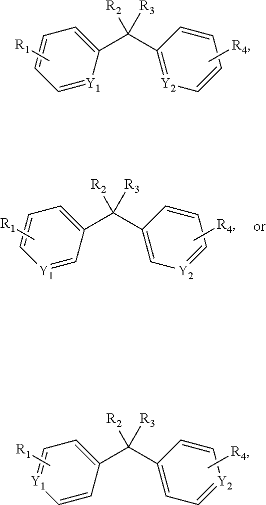

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂.

In some embodiments, the technology provides an insecticidal composition comprising a compound having a structure according to:

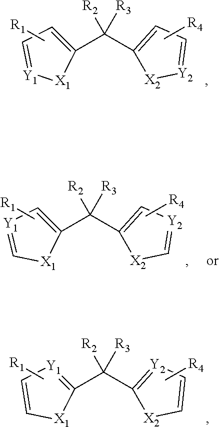

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, or S, wherein R₅ is H, alkyl, or aryl; and X₂ is CH—R₆, N—R₆, O, or S, wherein R₆ is H, alkyl, or aryl. In some embodiments, the compound is present at a weight percentage of from 0.1% to 99.9%.

In some embodiments, the technology provides use of a compound as described herein for the preparation of an insecticide. In some embodiments, the technology provides use of a compound as described herein for the preparation of an insecticide for killing an insect of the order Diptera, Lepidoptera, Coleoptera, or Hemiptera. In some embodiments, the technology provides use of a compound as described herein for the preparation of an insecticide for killing an insect of the superfamily Aphidoidea or of the family Culicidae, Noctuidae, Tortricidae, Crambidae, Erebidae, Chrysomelidae, or Aleyrodidae. In some embodiments, the technology provides use of a compound as described herein for the preparation of an insecticide for killing an insect of the genus Aedes, Anticarsia, Culex, Anopheles, Heliothis, Trichoplusia, Spodoptera, Chrysodeixis, Diatraea, Helicoverpa, Mythimna, Leptinotarsa, Diabrotica, or Chloridea. In some embodiments, the technology provides use of a compound as described herein for the preparation of an insecticide for killing an insect of a species that is Aedes aegypti, Culex quinquefasciatus, Heliothis viriscens, Trichoplusia ni, Spodoptera exigua, Chrysodends includens, Helicoverpa zea, Spodoptera eridania, Spodoptera frugiperda, Spodoptera mauritia, Spodoptera littoralis, Spodoptera litura, Diatraea saccharalls, Diatraea grandiosella, Anopheles quadrimaculatus, Leptinotarsa decemlineata, Diabrotica barberi, Diabtrotica virgifera, Diabrotica undecimpunctata, Phyllotreta cruciferae, Cerotoma trifurcate, or Anticarsia gemmetalls.

In some embodiments, the compound that finds use for the preparation of an insecticide comprises a structure according to:

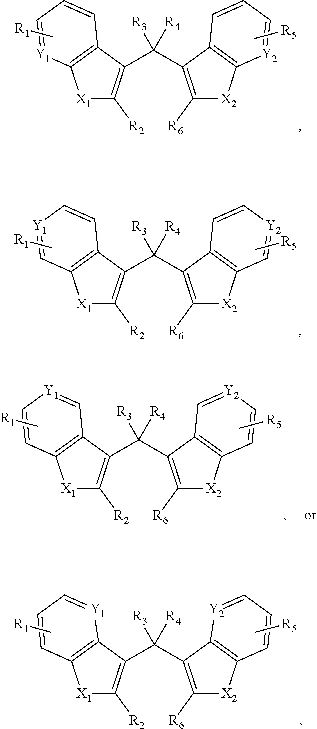

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃,or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl.

In some embodiments, the compound that finds use for the preparation of an insecticide comprises a structure according to:

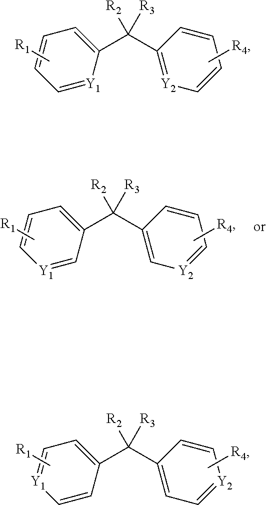

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂.

In some embodiments, the compound that finds use for the preparation of an insecticide comprises a structure according to:

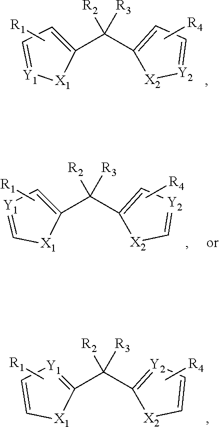

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, or S, wherein R₅ is H, alkyl, or aryl; and X₂ is CH—R₆, N—R₆, O, or S, wherein R₆ is H, alkyl, or aryl.

In some embodiments, the technology provides use of arundine for the synthesis of an insecticidal arundine analog compound.

Additional embodiments will be apparent to persons skilled in the relevant art based on the teachings contained herein.

### BRIEF DESCRIPTION OF THE DRAWINGS

These and other features, aspects, and advantages of the present technology will become better understood with reference to the following drawings.

FIG. 1A shows a schematic providing a generalized structure of active insecticidal compounds described herein, e.g., a compound (100) comprising a first heterocycle (130), a second heterocycle (140), a linker (e.g., a linker comprising a carbon atom) (120), and, optionally, a substituent (110) (e.g., a branching substituent).

FIG. 1B shows a general structure of a bisindole compound comprising a first indolyl (I1), a second indolyl (I2), and a linker (L) comprising n carbon atoms connecting the first indolyl to the second indolyl (I2).

FIG. 2A shows a scheme for synthesizing symmetrical bisindolylmethane compounds from a substituted indole (e.g., two or more equivalents) and a ketone or aldehyde. In some embodiments, R₁ is an alkyl, halogen, aryl, H, alkoxy, CN, CF₃, or NO₂; R₂ is an alkyl or H; R₃ is an alkyl, aryl, or H; and R₄ is an alkyl, aryl, or H.

FIG. 2B shows a scheme for synthesizing asymmetrical bisindolylmethane compounds from a substituted indole (e.g., provided in excess) and a ketone or aldehyde using a chiral catalyst (e.g., a chiral phosphoric acid catalyst). In some embodiments, R₁ is an alkyl, halogen, aryl, H, alkoxy, CN, CF₃, or NO₂; R₂ is an alkyl or H; R₃ is an alkyl, aryl, or H; R₄ is an alkyl, aryl, or H, R₅ is an alkyl, halogen, aryl, H, alkoxy, CN, CF₃, or NO₂; and R₆ is an alkyl or H.

FIG. 2C shows a scheme for synthesizing symmetrical bisindolylmethane compounds from a substituted indole (e.g., two or more equivalents) and a ketone or aldehyde.

FIG. 2D shows a scheme for synthesizing asymmetrical bisindolylmethane compounds from a substituted indole (e.g., provided in excess) and a ketone or aldehyde using a chiral catalyst (e.g., a chiral phosphoric acid catalyst).

FIG. 3A is a plot of data fit with a dose-response curve showing that the bisindole compound 3-((1H-indol-2-yl)methyl)-1H-indole is active against S. frugiperda.

FIG. 3B is a plot of data fit with a dose-response curve showing that the bisindole compound 3,3′-(pyridin-4-ylmethylene)bis(1H-indole) is active against S. frugiperda.

FIG. 3C is a plot of data showing that the bisindole compound 3,3′-diindolyl is not active against S. frugiperda.

FIG. 3D is a plot of data fit with a dose-response curve for killing of S. frugiperda by BIM-1 (arundine synthesized during the development of embodiments of the technology described herein, triangles). Data collected for killing by commercial arundine (Sigma) is provided as a control (circles).

In FIGS. 3E through 3NN, the test compound data are indicated by circles and the control compound data (BIM-1) are shown by triangles.

FIG. 3E is a plot of data fit with a dose-response curve for killing of S. frugiperda by BIM-2 (synthesized during the development of embodiments of the technology described herein). BIM-1 (arundine synthesized during the development of embodiments of the technology described herein) is shown as a control.

FIG. 3F is a plot of data fit with a dose-response curve for killing of S. frugiperda by BIM-3 (synthesized during the development of embodiments of the technology described herein). BIM-1 (arundine synthesized during the development of embodiments of the technology described herein) is shown as a control.

FIG. 3G is a plot of data fit with a dose-response curve for killing of S. frugiperda by BIM-4 (synthesized during the development of embodiments of the technology described herein). BIM-1 (arundine synthesized during the development of embodiments of the technology described herein) is shown as a control.

FIG. 3H is a plot of data fit with a dose-response curve for killing of S. frugiperda by BIM-5 (synthesized during the development of embodiments of the technology described herein). BIM-1 (arundine synthesized during the development of embodiments of the technology described herein) is shown as a control.

FIG. 3I is a plot of data fit with a dose-response curve for killing of S. frugiperda by BIM-6 (synthesized during the development of embodiments of the technology described herein). BIM-1 (arundine synthesized during the development of embodiments of the technology described herein) is shown as a control.

FIG. 3J is a plot of data fit with a dose-response curve for killing of S. frugiperda by BIM-7 (synthesized during the development of embodiments of the technology described herein). BIM-1 (arundine synthesized during the development of embodiments of the technology described herein) is shown as a control.

FIG. 3K is a plot of data fit with a dose-response curve for killing of S. frugiperda by BIM-8 (synthesized during the development of embodiments of the technology described herein). BIM-1 (arundine synthesized during the development of embodiments of the technology described herein) is shown as a control.

FIG. 3L is a plot of data fit with a dose-response curve for killing of S. frugiperda by BIM-9 (synthesized during the development of embodiments of the technology described herein). BIM-1 (arundine synthesized during the development of embodiments of the technology described herein) is shown as a control.

FIG. 3M is a plot of data fit with a dose-response curve for killing of S. frugiperda by BIM-10 (synthesized during the development of embodiments of the technology described herein). BIM-1 (arundine synthesized during the development of embodiments of the technology described herein) is shown as a control.

FIG. 3N is a plot of data fit with a dose-response curve for killing of S. frugiperda by BIM-11 (synthesized during the development of embodiments of the technology described herein). BIM-1 (arundine synthesized during the development of embodiments of the technology described herein) is shown as a control.

FIG. 3O is a plot of data fit with a dose-response curve for killing of S. frugiperda by BIM-12 (synthesized during the development of embodiments of the technology described herein). BIM-1 (arundine synthesized during the development of embodiments of the technology described herein) is shown as a control.

FIG. 3P is a plot of data fit with a dose-response curve for killing of S. frugiperda by BIM-13 (synthesized during the development of embodiments of the technology described herein). BIM-1 (arundine synthesized during the development of embodiments of the technology described herein) is shown as a control.

FIG. 3Q is a plot of data fit with a dose-response curve for killing of S. frugiperda by BIM-14 (synthesized during the development of embodiments of the technology described herein). BIM-1 (arundine synthesized during the development of embodiments of the technology described herein) is shown as a control.

FIG. 3R is a plot of data fit with a dose-response curve for killing of S. frugiperda by BIM-15 (synthesized during the development of embodiments of the technology described herein). BIM-1 (arundine synthesized during the development of embodiments of the technology described herein) is shown as a control.

FIG. 3S is a plot of data fit with a dose-response curve for killing of S. frugiperda by BIM-17 (synthesized during the development of embodiments of the technology described herein). BIM-1 (arundine synthesized during the development of embodiments of the technology described herein) is shown as a control.

FIG. 3T is a plot of data fit with a dose-response curve for killing of S. frugiperda by BIM-18 (synthesized during the development of embodiments of the technology described herein). BIM-1 (arundine synthesized during the development of embodiments of the technology described herein) is shown as a control.

FIG. 3U is a plot of data fit with a dose-response curve for killing of S. frugiperda by BIM-19 (synthesized during the development of embodiments of the technology described herein). BIM-1 (arundine synthesized during the development of embodiments of the technology described herein) is shown as a control.

FIG. 3V is a plot of data fit with a dose-response curve for killing of S. frugiperda by BIM-20 (synthesized during the development of embodiments of the technology described herein). BIM-1 (arundine synthesized during the development of embodiments of the technology described herein) is shown as a control.

FIG. 3W is a plot of data fit with a dose-response curve for killing of S. frugiperda by BIM-21 (synthesized during the development of embodiments of the technology described herein). BIM-1 (arundine synthesized during the development of embodiments of the technology described herein) is shown as a control.

FIG. 3X is a plot of data fit with a dose-response curve for killing of S. frugiperda by BIM-22 (synthesized during the development of embodiments of the technology described herein). BIM-1 (arundine synthesized during the development of embodiments of the technology described herein) is shown as a control.

FIG. 3Y is a plot of data fit with a dose-response curve for killing of S. frugiperda by BIM-23 (synthesized during the development of embodiments of the technology described herein). BIM-1 (arundine synthesized during the development of embodiments of the technology described herein) is shown as a control.

FIG. 3Z is a plot of data fit with a dose-response curve for killing of S. frugiperda by BIM-24 (synthesized during the development of embodiments of the technology described herein). BIM-1 (arundine synthesized during the development of embodiments of the technology described herein) is shown as a control.

FIG. 3AA is a plot of data fit with a dose-response curve for killing of S. frugiperda by BIM-25 (synthesized during the development of embodiments of the technology described herein). BIM-1 (arundine synthesized during the development of embodiments of the technology described herein) is shown as a control.

FIG. 3BB is a plot of data fit with a dose-response curve for killing of S. frugiperda by BIM-26 (synthesized during the development of embodiments of the technology described herein). BIM-1 (arundine synthesized during the development of embodiments of the technology described herein) is shown as a control.

FIG. 3CC is a plot of data fit with a dose-response curve for killing of S. frugiperda by BIM-27 (synthesized during the development of embodiments of the technology described herein). BIM-1 (arundine synthesized during the development of embodiments of the technology described herein) is shown as a control.

FIG. 3DD is a plot of data fit with a dose-response curve for killing of S. frugiperda by BIM-28 (synthesized during the development of embodiments of the technology described herein). BIM-1 (arundine synthesized during the development of embodiments of the technology described herein) is shown as a control.

FIG. 3EE is a plot of data fit with a dose-response curve for killing of S. frugiperda by BIM-29 methyl variant (synthesized during the development of embodiments of the technology described herein). BIM-1 (arundine synthesized during the development of embodiments of the technology described herein) is shown as a control.

FIG. 3FF is a plot of data fit with a dose-response curve for killing of S. frugiperda by BIM-30 (synthesized during the development of embodiments of the technology described herein). BIM-1 (arundine synthesized during the development of embodiments of the technology described herein) is shown as a control.

FIG. 3GG is a plot of data fit with a dose-response curve for killing of S. frugiperda by BIM-31 (synthesized during the development of embodiments of the technology described herein). BIM-1 (arundine synthesized during the development of embodiments of the technology described herein) is shown as a control.

FIG. 3HH is a plot of data fit with a dose-response curve for killing of S. frugiperda by BIM-32 (synthesized during the development of embodiments of the technology described herein). BIM-1 (arundine synthesized during the development of embodiments of the technology described herein) is shown as a control.

FIG. 3II is a plot of data fit with a dose-response curve for killing of S. frugiperda by BIM-33 (synthesized during the development of embodiments of the technology described herein). BIM-1 (arundine synthesized during the development of embodiments of the technology described herein) is shown as a control.

FIG. 3JJ is a plot of data fit with a dose-response curve for killing of S. frugiperda by BIM-34 (synthesized during the development of embodiments of the technology described herein). BIM-1 (arundine synthesized during the development of embodiments of the technology described herein) is shown as a control.

FIG. 3KK is a plot of data fit with a dose-response curve for killing of S. frugiperda by BIM-35 (synthesized during the development of embodiments of the technology described herein). BIM-1 (arundine synthesized during the development of embodiments of the technology described herein) is shown as a control.

FIG. 3LL is a plot of data fit with a dose-response curve for killing of S. frugiperda by BIM-36 (synthesized during the development of embodiments of the technology described herein). BIM-1 (arundine synthesized during the development of embodiments of the technology described herein) is shown as a control.

FIG. 3MM is a plot of data fit with a dose-response curve for killing of S. frugiperda by BIM-37 (RIS ratio 1 to 1) (synthesized during the development of embodiments of the technology described herein). BIM-1 (arundine synthesized during the development of embodiments of the technology described herein) is shown as a control.

FIG. 3NN is a plot of data fit with a dose-response curve for killing of S. frugiperda by BIM-37 (RIS ratio 3 to 1) (synthesized during the development of embodiments of the technology described herein). BIM-1 (arundine synthesized during the development of embodiments of the technology described herein) is shown as a control.

FIG. 4A shows the structure of arundine and exemplary structures of Series 1 arundine derivatives described herein in Example 3.

FIG. 4B shows the structure of exemplary structures of Series 2 arundine derivatives described herein in Example 3.

FIG. 4C shows the structure of exemplary structures of Series 3 arundine derivatives described herein in Example 3.

FIG. 4D shows the structure of exemplary structures of Series 4 arundine derivatives described herein in Example 3.

FIG. 4E shows the structure of exemplary structures of Series 5 arundine derivatives described herein in Example 3.

It is to be understood that the figures are not necessarily drawn to scale, nor are the objects in the figures necessarily drawn to scale in relationship to one another. The FIGS. are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to particular heterocyclic compounds that are analogs of arundine, including compounds that comprise structural modifications of arundine and substituted derivatives of arundine, and particularly, but not exclusively, to use of arundine analogs as an insecticide, methods of making arundine analogs, and methods of using arundine analogs for killing insects. In some embodiments, the technology relates to insecticidal compounds and particularly, but not exclusively, to bisindole compounds and derivatives thereof, methods of making bisindole compounds, and methods of using bisindole compounds and derivatives thereof for killing insects. In some embodiments, the bisindole compound is a bisindolylalkane. In some embodiments, the bisindolylalkane is a bisindolylmethane. In some embodiments, the bisindolylmethane is a derivative of arundine. In some embodiments, the bisindolylmethane is a derivative of 3-((1H-indo1-2-yl)methyl)-1H-indole.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

Where a term is provided in the singular, embodiments comprising the plural of that term are also provided. For example, unless otherwise required by context, singular terms shall include pluralities and plural terms shall include the singular.

As used herein, the terms “include,” “includes,” and “including” are to be construed as at least having the features to which they refer while not excluding any additional unspecified features.

As used herein, the term “insect” refers to any member of a large group of invertebrate animals characterized in the adult state by division of the body into head, thorax, and abdomen; three pairs of legs; and often (but not always) two pairs of membranous wings. This definition therefore includes but is not limited to a variety of biting insects (e.g., ants, bees, black flies, chiggers, fleas, green head flies, mosquitoes, stable flies, ticks, and wasps), wood-boring insects (e.g., termites), noxious insects (e.g., house flies, cockroaches, lice, roaches, and wood lice), and household pests (e.g., flour and bean beetles, dust mites, moths, silverfish, bed bugs, carpet beetles, furniture beetles, book lice, clothes moths, spiders and weevils). Other examples include locusts, caterpillars, bugs, hoppers, and aphids. This definition of “insect” also includes non-adult insect states including larva and pupa and thus, as used herein, the term “insect” refers to any stage of an insect life cycle (egg, larva, pupa, or adult).

As used herein, the term “egg” refers to both fertilized and unfertilized eggs. As used herein, the phrase “an insect inhibitory amount”, refers to an amount of a compound and/or composition that results in any measurable inhibition of insect motility, viability, growth, insect development, insect reproduction, insect egg laying, insect feeding behavior, insect mating behavior, and/or any measurable decrease in the adverse effects caused by insect feeding, egg laying or other interaction with a plant.

As used herein, the term “insecticidal activity” refers to the capacity of a compound and/or composition to kill, inhibit growth, inhibit reproduction, or otherwise negatively affect all or part of an insect pest. Accordingly, the term “insecticidal activity” refers to activity of a compound that can be measured by, but is not limited to, insect mortality, insect weight loss, insect repellency, and other behavioral and physical changes of an insect after feeding and/or exposure of the insect to the compound for an appropriate length of time. Thus, compound having insecticidal activity adversely impacts at least one measurable parameter of insect fitness. Complete lethality to insects is preferred but is not required to achieve functional activity. If, after exposure, an insect avoids the toxin or ceases feeding, that avoidance will be useful in some applications, even if the effects are sublethal or lethality is delayed or indirect.

As used herein, the term “heterologous” refers to any thing that is in a context other than that which it occurs in nature.

As used herein, the term “plant” is used in its broadest sense. It includes, but is not limited to, any species of grass (e.g., turf grass), ornamental or decorative, crop or cereal, fodder or forage, fruit or vegetable, fruit plant or vegetable plant, herb plant, woody plant, flower plant or tree. It is not meant to limit a plant to any particular structure. It also refers to a unicellular plant (e.g., microalga) and a plurality of plant cells that are largely differentiated into a colony (e.g., volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera. Transgenic plants may be produced using techniques known in the art.

As used herein, a “system” refers to a plurality of real and/or abstract components operating together for a common purpose. In some embodiments, each component of the system interacts with one or more other components and/or is related to one or more other components.

As used herein, the term “biological system” refers to a collection of genes, enzymes, activities, or functions that operate together to provide a metabolic pathway or metabolic network. A biological system may also be described in terms of nutrient flux, energy flux, electrochemical gradients, metabolic inputs (biological reactants), and metabolic outputs (biological products), e.g., that provide for conversion of energy inputs into energy for biological processes, anabolic synthesis of biomolecules, and elimination of wastes.

As used herein, the term “metabolic pathway” refers to a set of connected metabolic, biochemical, and physical processes that transform a metabolic input to a metabolic output in a series of steps and intermediates.

As used herein, the term “metabolic network” refers to a set of connected metabolic pathways. A metabolic network may transform a metabolic input to a metabolic output in a series of steps and intermediates.

As used herein, the term “naturally occurring” as applied to a molecule, compound, or composition, refers to a molecule, compound, or composition that is found in nature. For example, a molecule, compound, or composition that is produced by an organism that can be isolated from a source in nature and that has not been intentionally modified by a human in the laboratory is naturally occurring.

As used herein, the term “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reactions that occur within a natural environment.

Definitions of specific functional groups and chemical terms are described in more detail below. For purposes of this disclosure, the chemical elements are identified in accordance with the Periodic Table of the Elements, CAS version, Handbook of Chemistry and Physics, 75th edition, inside cover, and specific functional groups are generally defined as described therein. Additionally, general principles of organic chemistry, as well as specific functional moieties and reactivities, are described in Sorrell, Organic Chemistry, 2nd edition, University Science Books, Sausalito, 2006; Smith, March's Advanced Organic Chemistry: Reactions, Mechanism, and Structure, 7th edition, John Wiley & Sons, Inc., New York, 2013; Larock, Comprehensive Organic Transformations, 3rd edition, John Wiley & Sons, Inc., New York, 2018; Carruthers, Some Modern Methods of Organic Synthesis, 3rd edition, Cambridge University Press, Cambridge, 1987, the entire contents of each of which are incorporated herein by reference.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl. The term “Cₘ₋ₙ” is used interchangeably with “Cₘ-Cₙ” as described herein.

As used herein, the term “alkenyl” refers to a straight or branched hydrocarbon chain containing from 2 to 30 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” refers to a straight or branched hydrocarbon chain containing from 2 to 30 carbon atoms and containing at least one carbon-carbon triple bond. Representative examples of alkynyl include, but are not limited to, ethynyl, propynyl, and butynyl.

As used herein, the term “aryl” refers to a phenyl group or a bicyclic or tricyclic aromatic fused ring system. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl.

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cycloalkenyl” refers to a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl, and cycloheptenyl.

As used herein, the term “halogen” or “halo” means F, Cl, Br, or I.

As used herein, the term “haloalkyl” means an alkyl group, as defined herein, in which one, two, three, four, five, six, seven, or eight hydrogen atoms are replaced by a halogen.

As used herein, the term “heteroaryl” refers to an aromatic monocyclic ring or an aromatic bicyclic ring system or an aromatic tricyclic ring system. The aromatic monocyclic rings are five-membered or six-membered rings containing at least one heteroatom independently selected from the group consisting of N, O, and S (e.g., 1, 2, 3, or 4 heteroatoms independently selected from O, S, and N). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic, heteroaryl group, as defined herein. The tricyclic heteroaryl groups are exemplified by a monocyclic heteroaryl ring fused to two rings independently selected from a monocyclic aryl group, as defined herein, or a monocyclic heteroaryl group, as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” means a monocyclic heterocycle, a bicyclic heterocycle, or a tricyclic heterocycle. The monocyclic heterocycle is a three-membered, four-membered, five-membered, six-membered, seven-membered, or eight-membered ring containing at least one heteroatom independently selected from the group consisting of O, N, and S. The three-membered or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one, or two double bonds and one, two, or three heteroatoms selected from the group consisting of O, N, and S. The seven-membered and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of 2, 3, or 4 carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

As used herein, the term “hydroxy” means an —OH group.

In some instances, the number of carbon atoms in a group (e.g., alkyl, alkoxy, or cycloalkyl) is indicated by the prefix “Cₓ-C_{y}-”, wherein x is the minimum and y is the maximum number of carbon atoms in the group. Thus, for example, “C₁-C₃-alkyl” refers to an alkyl group containing from 1 to 3 carbon atoms.

As used herein, the term “substituent” refers to a group substituted on an atom of the indicated group. When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable group known to those of skill in the art (e.g., one or more of the groups recited below). Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl. For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

As used herein, the “LC₅₀” of a compound for an insect refers to a concentration of the compound that kills 50% of the insects of a tested population contacted with the chemical during the observation period. A lower LC₅₀ is indicative of increased toxicity of the compound for the insect.

As used herein, the term “contacts” when used in reference to a compound contacting an insect may refer to any mode of the compound contacting the insect, e.g., by the insect ingesting the compound, by the compound contacting an external surface of the insect, by the compound penetrating through the insect cuticle or exoskeleton, by the compound entering through the insect spiracle, etc.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however, of any latent ambiguity, definitions provided herein take precedent over any dictionary or extrinsic definition.

### Description

Provided herein is technology relating to particular heterocyclic compounds that are analogs of arundine, including compounds that comprise structural modifications of arundine and substituted derivatives of arundine, and particularly, but not exclusively, to use of arundine analogs as an insecticide, methods of making arundine analogs, and methods of using arundine analogs for killing insects. In some embodiments, the technology provides arundine analogs (e.g., bisindole compounds), insecticidal compositions (e.g., compositions comprising insecticidal arundine analogs), and methods of using arundine analogs and insecticidal compositions comprising arundine analogs to kill insects. In some embodiments, the technology relates to use of a bisindole (e.g., bisindolylalkane (e.g., bisindolylmethane (e.g., arundine))) as a chemical scaffold that is modified by chemical synthetic and/or biochemical enzymatic methods to produce insecticidal compounds, insecticidal compositions comprising insecticidal compounds, and methods of using an insecticidal compound or insecticidal composition to kill an insect.

### Compounds

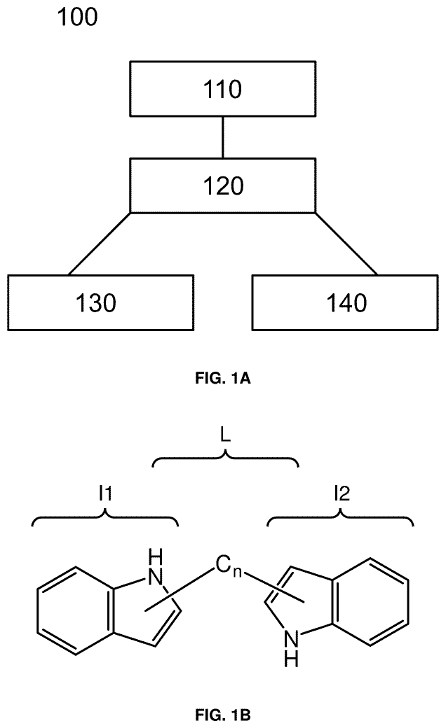

In some embodiments, the technology relates to arundine analogs. For example, in some embodiments, the technology provides a compound (100) comprising a first heterocycle (130), a second heterocycle (140), a linker (e.g., a linker comprising a carbon atom) (120), and, optionally, a branching substituent (110) (see FIG. 1A). As discussed further herein, compounds having the structure shown in FIG. 1A have insecticidal activity as described hereinbelow. In some embodiments, the technology provides heterocyclic compounds, e.g., arundine analogs that are bisindole compounds derived from 3,3-diindolylmethane (arundine) or 34(1H indol-2-yl)methyl)-1H-indole.

As used herein, the term “arundine analog” refers to a compound that is a substituted derivative of 3,3′-diindolylmethane or 3-((1H-Indol-2-yl)methyl)-1H-indole; a compound that comprises a structure resulting from a structural modification of a heterocycle of 3,3′-diindolylmethane or 3((1H-Indol-2-yl)methyl)-1H-indole; or a compound that comprises a structural modification of a linker joining the heterocycles of 3,3′-diindolylmethane or 3-((1H-Indol-2-yl)methyl)-1H-indole.

As used herein, the term “structural modification” of a heterocycle refers to modifying the chemical structure of a heterocycle by adding an atom (e.g., increasing ring size), removing an atom (e.g., decreasing ring size), and/or replacing an atom with an atom of another type (e.g., replacing C with N, O, or S; replacing N with C, O, or S; replacing O with N, C, or S; or replacing S with C, N, or O). As used herein, the term “structural modification” of a linker refers to modifying the chemical structure of a linker by adding an atom (e.g., increasing linker size), removing an atom (e.g., decreasing linker size), and/or replacing an atom with an atom of another type (e.g., replacing C with N, O, or S; replacing N with C, O, or S; replacing O with N, C, or S; or replacing S with C, N, or O).

In some embodiments, an arundine analog is a bisindole compound. As used herein, the term “bisindole compound” refers to a chemical compound comprising two indolyl moieties (I1 and I2) and a linker (L) connecting the pyrrolyl portions of the indolyl groups, e.g. (see also FIG. 1B):

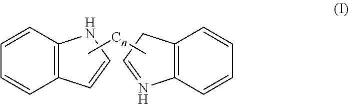

In some embodiments, L is a linker having n atoms. In some embodiments, L is a linker having n carbon atoms. In some embodiments, n is at least 1 (e.g., 1, 2, 3, 4, 5, 6, 7, 8, 9, or 10). In some embodiments, n is 1 and the bisindole compound is a bisindolylmethane.

In some embodiments, the technology provides an analog of 3,3′-diindolylmethane (arundine, CAS 1968-05-4) or a bisindole compound that is derived from 3,3′-diindolylmethane, e.g., by modifying one or more rings of the indolyl groups and/or by adding substituents to the linker and/or indolyl groups of 3,3′-diindolylmethane:

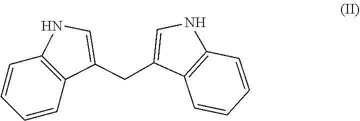 [SMILES: c1ccc2c(Cc3c[nH]c4ccccc34)c[nH]c2c1]

In some embodiments, the technology provides an analog of 3-((1H-Indol-2-yl)methyl)-1H-indole (CAS 114648-66-7) or a bisindole compound that is derived from 3-((1H-Indol-2-yl)methyl)-1H-indole, e.g., by modifying one or more rings of the indolyl groups and/or by adding substituents to the linker and/or indolyl groups of 3-((1H-Indol-2-yl)methyl)-1H-indole:

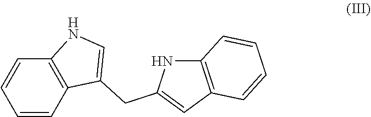 [SMILES: c1ccc2[nH]c(Cc3c[nH]c4ccccc34)cc2c1]

As described further herein, in some embodiments, the technology provides compounds in which an arundine analog (e.g., bisindole compound) comprises substituents at any of the indolyl ring positions and/or comprises substituents attached to the linker.

As described further herein, in some embodiments, the technology provides compounds in which an arundine analog has a structure comprising structural modifications of arundine heterocyclic rings.

In some embodiments, the technology provides compounds comprising a bisindolylmethane moiety comprising one or more substituents on one or both indolyls and/or on the linker. The technology provides other modifications of the bisindolylmethane structure as discussed further below. For example, embodiments of the technology provide bisindolylmethane compounds that are modified as described herein to provide a compound having insecticidal activity.

In some embodiments, compounds described herein comprise three components: 1) a heterocycle; 2) a central linker (e.g., a central carbon linker); and 3) a branching substituent (FIG. 1A and FIG. 1B). In some embodiments, one or more structural modifications are made to one, two, or three of these components to provide desired physiochemical properties and bioactivities (e.g., killing) of the molecules and/or to modify (e.g., improve) physiochemical properties and bioactivities (e.g., killing).

For example, embodiments provide compounds comprising a heterocycle comprising nitrogen, a heterocycle comprising oxygen, and/or a heterocycle comprising sulfur (e.g., to provide a nitrogen-containing, an oxygen-containing, and/or a sulfur-containing heterocycle). In some embodiments, compounds comprise a heteroatom (e.g., nitrogen, oxygen, or sulfur) at any position within the heterocycle ring(s). In some embodiments, one or more heterocycles comprises one or more substituents (e.g., alkyl, aryl, substituted aryl, amino, hydroxyl, thiol, nitro, cyano, halogen, amido, ether, ester, carboxyl, and derivatives thereof). In some embodiments, compounds comprise one, two, three, or four heterocycles.

Further, embodiments provide compounds comprising a central carbon linker that links the two heterocycles. In some embodiments, compounds comprise a central carbon linker attached to one heterocycle and thus, in such compounds, the linker does not link two moieties but provides a substituent for the heterocycle to which it is attached. In some embodiments, the central carbon linker comprises a number of non-hydrogen branching substituents.

In some embodiments, the branching component comprises a substituent that is an alkyl, aryl, or a substituted alkyl and aryl, including heterocyclic substituents.

In some embodiments, the compounds provided herein are symmetric. In some embodiments, the compounds provided herein are asymmetric and comprise a chiral center at the central carbon linker.

Accordingly, in some embodiments, the technology provides a compound according to (IV):

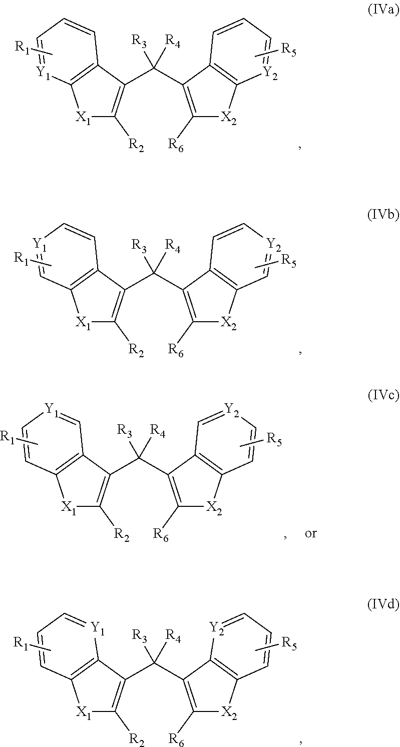

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₃ is H, alkyl, or aryl; R₄ is H, alkyl, or aryl; R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₇, N—R₇, O, or S, wherein R₇ is H, alkyl, or aryl; and X₂ is CH—R₈, N—R₈, O, or S, wherein R₈ is H, alkyl, or aryl. Furthermore, in various embodiments, the number and/or identity of substituents (R₁, R₂, R₃, R₄, R₅, R₆, R₇, R₈, X₁, X₂, Y₁, and/or Y₂) at indicated positions can be varied and can include multiple substituents (e.g., one at each position) within a molecule.

In some embodiments, the technology provides a compound according to (V):

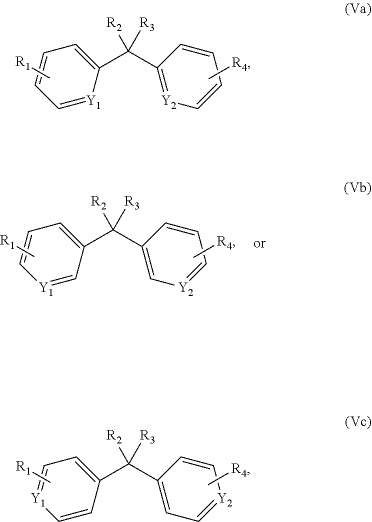

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ H, alkyl, or aryl; and R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂. Furthermore, in various embodiments, the number and/or identity of substituents (R₁, R₂, R₃, R₄, Y₁, and/or Y₂) at indicated positions can be varied and can include multiple substituents (e.g., one at each position) within a molecule.

In some embodiments, the technology provides a compound according to (VI):

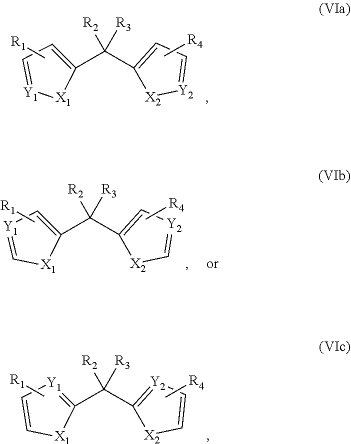

wherein: Y₁ is N or C; Y₂ is N or C; R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; R₂ is H, alkyl, or aryl; R₃ is H, alkyl, or aryl; R₄ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂; X₁ is CH—R₅, N—R₅, O, S, wherein R₅ is H, alkyl, or aryl; and X₂ is CH—R₆, N—R₆, O, or S, wherein R₆ is H, alkyl, or aryl. Furthermore, in various embodiments, the number and/or identity of substituents (R₁, R₂, R₃, R₄, R₅, R₆, X₁, X₂, Y₁, and/or Y₂) at indicated positions can be varied and can include multiple substituents (e.g., one at each position) within a molecule.

During the development of embodiments of the technology provided herein, data were collected that indicated that a compound comprising two indole groups joined by at least a single carbon central linker has potent insecticidal activity. In particular, data indicated that monoindoles, trisindoles, and tetraindoles had reduced and/or minimal killing activity, and compounds that did not comprise a carbon linker joining the two indole rings had reduced and/or minimal killing activity. Further, data indicated that halogenation, alkylation, and hydroxylation of the indole rings is tolerated—in some cases, data indicated that modification of the indole ring (e.g., fluorination at position 5 of the indole) improves insecticidal activity. Data indicated that branching (e.g., alkylation and acylation) at the central carbon linker is tolerated. These preliminary structure-activity relationships suggest that further modifications of the bisindolylmethane backbone can be made to improve the physiochemical and insecticidal activities of the molecules.

### Insecticidal Compositions

In some embodiments, the technology provides a composition comprising, consisting of, or consisting essentially of a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc. In some embodiments, the technology provides a composition comprising, consisting of, or consisting essentially of an insecticidal compound that is an arundine analog (e.g., a bisindole compound) described herein. In some embodiments, the composition comprises a plurality of arundine analogs (e.g., 2, 3, 4, 5, 6, 7, 8, 9, 10, or more different arundine analogs) described herein. In some embodiments, compositions include varying amounts of other formulation adjuvants, such as, for example, surfactants (e.g., non-ionic, anionic, cationic, and zwitterionic surfactants); fatty acids and fatty acid esters (e.g., alkyl palmitate, alkyl oleate, alkyl linoleate); and other auxiliary ingredients such as, for example, emulsifiers, dispersants, stabilizers, suspending agents, penetrants, coloring agents and/or dyes, and fragrances, as necessary or desired, e.g., as further described below. Components other than the insecticidal compound can be included in the compositions in any amount as long as the composition has some amount of insecticidal efficacy. In some embodiments, formulations are prepared, e.g., by mixing the insecticidal compound with formulation adjuvants to obtain compositions in the form of finely divided solids, granules, solutions, dispersions, or emulsions. The insecticidal compound can also be formulated with other adjuvants, such as finely divided solids, mineral oils, oils of vegetable or animal origin, modified oils of vegetable or animal origin, organic solvents, water, surface-active substances, or combinations thereof. Further adjuvants that can be used in insecticidal formulations include crystallization inhibitors, viscosity modifiers, suspending agents, dyes, anti-oxidants, foaming agents, light absorbers, mixing auxiliaries, antifoams, complexing agents, neutralizing or pH-modifying substances and buffers, corrosion inhibitors, fragrances, wetting agents, take-up enhancers, micro-nutrients, plasticizers, glidants, lubricants, dispersants, thickeners, antifreezes, microbicides, and liquid and solid fertilizers.

In some embodiments, compositions generally comprise from 0.1 to 99% by weight, especially from 0.1 to 95% by weight, of a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; and from 1 to 99.9% by weight of a formulation adjuvant, which, in some embodiments, includes from 0 to 25% by weight of a surface-active substance. In some embodiments, compositions generally comprise from 0.1 to 99% by weight, especially from 0.1 to 95% by weight, of an arundine analog and from 1 to 99.9% by weight of a formulation adjuvant, which, in some embodiments, includes from 0 to 25% by weight of a surface-active substance. Whereas commercial products may preferably be formulated as concentrates, the end user will normally employ dilute formulations. In some embodiments, the technology provides compositions comprising approximately 0.01 to 99.9% by weight of a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc. In some embodiments, the technology provides compositions comprising approximately 0.01 to 99.9% by weight of an arundine analog. In some embodiments, the technology provides compositions comprising 10.0%, 15.0%, 20.0%, 25.0%, 30.0%, 35.0%, 40.0%, 45.0%, 50.0%, 55.0%, 60.0%, 65.0%, 70.0%, 75.0%, 80.0%, 85.0%, or 90.0% by weight of an arundine analog.

As used herein, the term “arundine analog insecticide” refers to an arundine analog that has insecticidal activity, a composition comprising an arundine analog that has insecticidal activity, an arundine analog that finds use as an insecticide, or to a composition comprising an arundine analog that finds use as an insecticide. In some embodiments, the technology provides compositions comprising a “second insecticide” that is not a an arundine analog. As used herein, the term “insecticide” may refer to an arundine analog insecticide, a composition comprising an arundine analog insecticide, a second insecticide, or to a composition comprising one or more arundine analog insecticide(s) and one or more second insecticide(s).

The amount of insecticide (e.g., arundine analog insecticide and/or second insecticide) in the composition can range broadly and can depend on the particular agent as well as the intended use of the composition (e.g., based on method of application and/or particular target insect). While the amount of insecticide can each range broadly, for a composition to be registered and marketed as a “pesticide” within the United States for some uses (e.g., public health uses and pest control in residential structures), the Environmental Protection Agency (EPA) requires that a composition exhibit a 95% insect mortality at the lowest labeled rate. The EPA also regulates the upper limits of active agent(s) that can be used in practice in the environment. Thus, in some embodiments, the compositions provided herein comprise an amount (e.g., weight %) of an insecticide in a range that allows for at least some degree of insecticidal efficacy when the composition is used, while not necessarily meeting the EPA requirements for an insecticide for certain uses (i.e., more than 0%, but less than 95% insect mortality rate). In some embodiments, the amount (e.g., weight %) of the insecticide in the composition meets or exceeds the EPA requirements for an insecticide suitable for certain uses and in certain applications (e.g., sold as a concentrate or ready-to-use product).

One skilled in the art can select an appropriate amount of the insecticide (e.g., arundine analog insecticide and/or second insecticide) depending on the type of insect as well as the particular method of application. In exemplary embodiments, an amount of the insecticide can be selected such that the composition balances the insecticidal efficacy with the cost of the insecticide as well as balance risk of undesirable side effects (e.g., animal (fish or mammal) toxicity and/or environmental impact).

In some embodiments, compositions provided herein comprise a total amount of insecticide that is at least approximately 2.5% by weight, at least approximately 3.0% by weight, at least approximately 3.5% by weight, at least approximately 4.0% by weight, at least approximately 5.0% by weight, at least approximately 6.0% by weight, or at least approximately 7.5% by weight. In other embodiments, compositions provided herein comprise a total amount of insecticide that is no more than approximately 10.0% by weight, no more than approximately 7.5% by weight, no more than approximately 6.0% by weight, no more than approximately 5.0% by weight, or no more than approximately 4.0% by weight. In some embodiments, compositions provided herein comprise a total amount of insecticide that is approximately 2.5% by weight to approximately 10% by weight.

In some embodiments, the composition comprises from approximately 15 to approximately 40 percent by weight of an arundine analog. In some embodiments, the composition comprises at least approximately 15% by weight, or at least approximately 20% by weight, or at least approximately 25% by weight, or at least approximately 30% by weight, or at least approximately 35% by weight of an arundine analog. In some embodiments, the compositions comprise no more than approximately 40% by weight, or no more than approximately 35% by weight, or no more than approximately 30% by weight, or no more than approximately 25% by weight of an arundine analog.

In some embodiments, compositions are generally formulated in various ways (e.g., to provide a formulation) using formulation adjuvants, such as carriers, solvents, and surface-active substances (e.g., wetting agents). The formulations can be in various physical forms, e.g. in the form of dusting powders, gels, wettable powders, water-dispersible granules, water-dispersible tablets, effervescent pellets, emulsifiable concentrates, micro-emulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films, or in other forms known in the art, e.g., from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilizers, micronutrients, biological organisms, oil, or solvents.

In some embodiments, compositions are provided that comprise a formulation adjuvant (e.g., to provide a formulation). In some embodiments, formulations comprise one or more carriers and/or diluents (e.g., a solid or liquid carrier or diluent that finds use in pesticidal, insecticidal, agricultural, or horticultural compositions). In some embodiments, a liquid carrier comprises or is water, toluene, xylene, petroleum ether, vegetable oil, acetone, methyl ethyl ketone, cyclohexanone, acid anhydride, acetonitrile, acetophenone, amyl acetate, 2-butanone, butylene carbonate, chlorobenzene, cyclohexane, cyclohexanol, an alkyl ester of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and/or an alcohol of higher molecular weight, such as amyl alcohol, tetrahydro-furfuryl alcohol, hexanol, octanol, ethylene glycol, propylene glycol, glycerol, N-methyl-2-pyrrolidone, and the like. In some embodiments, carriers and diluents can include, for example, solvents (e.g., water, alcohols, acids, and esters).

In some embodiments, compositions include an additive comprising an oil of vegetable or animal origin, a mineral oil, alkyl esters of such oils, or mixtures of such oils and oil derivatives. The amount of oil additive in the composition is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. In some embodiments, compositions comprise an oil that is a vegetable and/or plant-based oil and/or an ester derivative thereof (e.g., wintergreen oil, cedarwood oil, rosemary oil, peppermint oil, geraniol, rose oil, palmarosa oil, citronella oil, citrus oils (e.g., lemon oil, lime oil, and orange oil), dillweed oil, corn oil, sesame oil, cottonseed oil, safflower oil, wheat germ oil, pine oil, cormint oil, soybean oil, palm oil, vegetable oil, olive oil, peanut oil, and canola oil). In some embodiments, oil additives comprise alkyl esters of C₈ to C₂₂ fatty acids, especially the methyl derivatives of C₁₂ to C₁₈ fatty acids, for example, the methyl esters of lauric acid, palmitic acid, and oleic acid (methyl laurate, methyl palmitate, and methyl oleate, respectively). Many oil derivatives are known from the Compendium of Herbicide Adjuvants, 10th Edition, Southern Illinois University, 2010, which is incorporated herein by reference.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin, and similar substances.

Embodiments provide that the carrier or diluent does not reduce (e.g., does not effectively and/or does not substantially reduce) the insecticidal efficacy of the formulation relative to the efficacy of the formulation in the absence of the additional component. In some embodiments, the carrier or diluent affects the physical characteristics of the formulation such that the formulation has a desired physical profile.

In some embodiments, formulations provided herein comprise a surface-active agent (e.g., a wetting agent). Surface-active agents are known to have various properties or characteristics, such as the ability to lower the surface tension between two components of a liquid composition or to improve wetting of a liquid on a surface. In some embodiments, a surface-active agent provided in a formulation enhances efficacy of an insecticidal formulation, e.g., by improving the penetration of an insecticide though an insect cuticle (e.g., providing an additional route of entry for an insecticide by facilitating the absorption or penetration of the insecticide though the insect cuticle, thus improving and/or maximizing insect mortality over a given exposure time). A large number of surface-active substances (e.g., wetting agents) can advantageously be used in both solid and liquid formulations, especially in those formulations that are diluted with a carrier prior to use. Surface-active substances may be anionic, cationic, non-ionic, or polymeric and they can be used as emulsifiers, wetting agents, or suspending agents or for other purposes. Typical surface-active substances include, for example, salts of alkyl sulfates, such as diethanolammonium lauryl sulfate salts of alkylarylsulfonates, such as calcium dodecyl-benzenesulfonate alkylphenol/alkylene oxide addition products, such as nonylphenol ethoxylate alcohol/alkylene oxide addition products, such as tridecylalcohol ethoxylate soaps, such as sodium stearate salts of alkylnaphthalenesulfonates, such as sodium dibutylnaphthalenesulfonate dialkyl esters of sulfosuccinate salts, such as sodium di(2-ethylhexyl)sulfosuccinate sorbitol esters, such as sorbitol oleate quaternary amines, such as lauryltrimethylammonium chloride, polyethylene glycol esters of fatty acids, such as polyethylene glycol stearate block copolymers of ethylene oxide and propylene oxide, and salts of mono- and di-alkylphosphate esters and also further substances described, e.g., in McCutcheon's Detergents and Emulsifiers Annual, MC Publishing Corp., Ridgewood New Jersey (1981).

In some embodiments, the composition can be formulated (e.g., to provide a formulation) for application or delivery as an aerosol or a fog wherein the composition allows for the formation of droplets having an average diameter of approximately 1 μm to approximately 30 μm (e.g., 1, 2, 3, 4, 5, 6, 7, 8, 9, 10, 11, 12, 13, 14, 15, 16, 17, 18, 19, 20, 21, 30 22, 23, 24, 25, 26, 27, 28, 29, or 30 μm). Suitable compositions for such a formulation typically have a viscosity that allows for the composition to atomize without clogging an application nozzle. Such viscosities can vary and be readily determined by one of skill in the art; however, a non-limiting common minimum viscosity is approximately 1 centistokes (cts). In some embodiments, the formulation comprises a concentration of insecticide (e.g., arundine analog insecticide and, optionally, a second insecticide) that is adequate for insecticidal activity when applied in a volume from approximately 0.3 to approximately 2.0 fluid ounces per acre (e.g., an ultra-low volume (ULV) application).

In some embodiments, an arundine analog described herein is contained in very fine microcapsules. Microcapsules contain the arundine analog in a porous carrier. This enables the arundine analog to be released into the environment in controlled amounts (e.g., slow release). Microcapsules usually have a diameter of from 0.1 to 500 microns. In some embodiments, they contain arundine analog in an amount of approximately from 25 to 95% by weight of the capsule weight. The arundine analog can be in the form of a monolithic solid, in the form of fine particles in solid or liquid dispersion, or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane, chemically modified polymers and starch xanthates, or other polymers that are known to the person skilled in the art. Alternatively, very fine microcapsules can be formed in which the arundine analog is contained in the form of finely divided particles in a solid matrix of base substance, but the microcapsules are not themselves encapsulated.

Embodiments include commercially useful formulations or “ready-to-use” application forms. In such formulations, a composition of arundine analog can be provided as a mixture with other active compounds, for example, various additional insecticides, pesticides, fungicides, antimicrobials, and/or herbicides, as well as plant growth regulators, insect repellents, attractants, fertilizers, and/or fragrances, to expand the applicability of insecticidal compositions described herein. Embodiments provide for the compositions to be manufactured as formulations that are useful for insect control. In some embodiments, the composition can be formulated as an emulsion, a liquid concentrate, a sol (flowable agent), an aerosol (e.g., a fogger), a liquid for ultra-low volume (ULV) application, or the like, by any standard or conventional methods for mixing and manufacturing such formulations such as, for example, admixing arundine analog with any suitable additional inert ingredient that is used as a carrier, solvent, diluent, emulsifier, dispersant, stabilizer, suspending agent, or penetrant. The addition of these materials would depend on the arundine analog and the type of formulation and how it is intended to be applied.

In some embodiments, formulations comprise one or more compounds that increases the long-term stability of the insecticide(s) in the formulation. Thus, some embodiments comprise an antioxidant (e.g., to provide stabilization to oxidation) and/or a UV light absorber (e.g., to provide stabilization to light exposure). Such compounds are known in the art. Packaging and/or storage containers for the compositions and/or formulations described herein can be selected to provide protection from degradation of actives by oxygen and light exposure (e.g., vacuum packaging, inert atmosphere, deoxygenated solvents, and opaque/colored containers).

In some embodiments, a composition is in the form of a concentrate that is diluted prior to use, although ready-to-use compositions can also be made. The final dilution is usually made with water, but can be made instead of, or in addition to, water, with, for example, liquid fertilizers, micronutrients, biological organisms, oil, or solvents.

### Methods of Using Insecticidal Compounds

In some embodiments, the technology described herein provides a method for insect control comprising contacting an insect (e.g., by ingestion, by external contact, by penetration through a cuticle or exoskeleton, by entrance through a spiracle, etc.) with an amount of any of the compositions and/or formulations herein described (e.g., a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; arundine analogs (e.g., bisindole compounds); and compounds and/or formulations comprising an arundine analog (e.g., a bisindole compound)). In some embodiments, the method comprises contacting (e.g., by ingestion, by external contact, by penetration through a cuticle or exoskeleton, by entrance through a spiracle, etc.) an insect with an amount of a composition comprising, consisting essentially of, or consisting of an arundine analog described herein. In some embodiments, contacting (e.g., by ingestion, by external contact, by penetration through a cuticle or exoskeleton, by entrance through a spiracle, etc.) a population of insects with an amount of a composition comprising, consisting essentially of, or consisting of an arundine analog kills approximately 95% of the contacted insect population.

In some embodiments, the method comprises contacting (e.g., by ingestion, by external contact, by penetration through a cuticle or exoskeleton, by entrance through a spiracle, etc.) an insect with an amount of a composition comprising, consisting essentially of, or consisting of an arundine analog that is effective to provide approximately 95% insect mortality within 24 hours at the lowest labeled rate (per EPA guideline).

In some embodiments, the methods described herein can comprise any known route, apparatus, and/or mechanism for the delivery or application of the compositions and formulations. In some embodiments, the method comprises spraying (e.g., using a sprayer). Traditional pesticide sprayers are typically operated manually or electrically or are gas-controlled and use maximum pressures ranging from 15 to 500 psi to generate flow rates from 5 gpm to 40 gpm. In some embodiments, the methods disclosed herein comprise the use of the compositions and/or formulations in combination with any low volume environmental pest control device(s) such as, for example, ultra-low volume (ULV) machines. Such combinations are useful in methods for insect control wherein contacting the insect with a low volume of the composition is possible and/or desirable. ULV machines use low volume of material, for example, at rates of approximately one gallon per hour (or ounces per minute), and typically utilize artificial wind velocities such as from, for example, an air source (e.g., pump or compressor) to break down and distribute the composition/formulation into a cold fog (e.g., having average droplet particle sizes of approximately 1-30 μm).

The rates of application vary within wide limits and depend on the pest to be controlled, the prevailing climatic conditions, and other factors governed by the method of application and the time of application. For example, embodiments provide application at a rate of 0.0002-200 pounds of active ingredient/acre (lbs a.i./A). In some embodiments, the rate of application is adequate to provide an insect inhibitory amount of an arundine analog insecticide to contact insects in the area of application.

In some embodiments, methods comprise applying a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; a composition comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; and/or a formulation comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc to a plant. In some embodiments, methods comprise applying a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; a composition comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; and/or a formulation comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc to a textile (e.g., an article of clothing, a tent). In some embodiments, methods comprise applying methods comprise applying a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; a composition comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; and/or a formulation comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc to a surface. In some embodiments, methods comprise applying methods comprise applying a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; a composition comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc; and/or a formulation comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc to a mammal (e.g., a human), e.g., to the skin or hair of the mammal.

In some embodiments, methods comprise applying a compound (e.g., arundine analog (e.g., a bisindole compound)), composition, and/or formulation herein described to a plant. In some embodiments, methods comprise applying a compound (e.g., arundine analog (e.g., a bisindole compound)), composition, and/or formulation herein described to a textile (e.g., an article of clothing, a tent). In some embodiments, methods comprise applying a compound (e.g., arundine analog (e.g., a bisindole compound)), composition, and/or formulation herein described to a surface. In some embodiments, methods comprise applying a compound (e.g., arundine analog (e.g., a bisindole compound)), composition, and/or formulation herein described to a mammal (e.g., a human), e.g., to the skin or hair of the mammal.

### Insect Killing Assay Methods

Methods for measuring insecticidal activity are well known in the art. See, for example, Czapla and Lang, (1990) J. Econ. Entomol. 83: 2480-2485; Andrews, et al., (1988) Biochem. J. 252: 199-206; Marrone, et al., (1985) J. of Econ. Entomol. 78: 290-293 and U.S. Pat. No. 5,743,477, each of which is incorporated herein by reference. Generally, a compound is mixed and used in feeding and/or external contact assays. See, for example Marrone, et al., (1985) J. of Economic Entomology 78: 290-293, which is incorporated herein by reference. Such assays can include contacting an insect with one or more compounds and determining the ability of the compound to cause the death of the insect. For example, the ability of a compound to cause the death of an insect comprises, in some embodiments, contacting a number of insects with a composition and counting the number of dead insects as a fraction of total insects contacted and/or as a function of time. In some embodiments, the ability of a compound to cause the death of an insect comprises contacting a number of insects with a series of known concentrations of the composition (e.g., a serial dilution series such as that described in Example 1 and in Example 2) and determining a concentration of the compound that kills 50% of the insects. In some embodiments, methods for measuring insecticidal activity are used to measure the insecticidal activity of an arundine analog, a composition comprising an arundine analog, or a formulation comprising an arundine analog.

### Insects

In some embodiments, a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc (e.g., arundine analogs (e.g., bisindole compounds)), compositions comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc (e.g., arundine analogs (e.g., bisindole compounds)), and formulations comprising a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc (e.g., arundine analogs (e.g., bisindole compounds)) as disclosed herein find use as an insecticide.

When an insect is the target pest for the present technology, such pests include but are not limited to: from the order Lepidoptera, for example, Acleris spp., Adoxophyes spp., Aegeria spp., Agrotis spp., Alabama argillaceae, Amnylois spp., Anticarsia gemmatalis, Archips spp, Argyrotaenia spp., Autographa spp., Busseola fusca, Cadra cautella, Carposina nipponensis, Chzlo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Mythimna spp., Operophtera spp., Ostrinia Nubzlalzs, Pammene spp., Pandemis spp., Pariahs flammea, Pectinophora gossypiella, Phthorinaea operculella, Pieris rapae, Pieris spp., Plutella xylostella, Prays spp., Scirpophaga spp., Sesamia spp., Sparganothis spp., Spodoptera spp. (e.g., Spodoptera mauritia, Spodoptera littoralis, Spodoptera litura), Synanthedon spp., Thawnetopoea spp., Tortrix spp., Trichoplusia ni and Yponomeuta spp.; from the order Coleoptera, for example, Agriotes spp., Anthonomus spp., Atomaria linearis, Cerotoma spp. (e.g., C. trifurcata), Chaetocnema tibialis, Cosmopolites spp., Curculio spp., Dennestes spp., Diabrotica spp. (e.g., Diabrotica barberi, Diabtrotica virgifera, Diabrotica undecimpunctata), Epilachna spp., Eremnus spp., Leptinotarsa decemlineata, Lissorhoptrus spp., Melolontha spp., Orycaephilus spp., Otiorhynchus spp., Phlyctinus spp., Phyllotreta spp. (e.g., P. cruciferae), Popillia spp., Psylliodes spp., Rhizopertha spp., Scarabeidae, Sitophilus spp., Sitotroga spp., Tenebrio spp., Tribolium spp. and Trogoderna spp.; from the order Orthoptera, for example, Blatta spp., Blattella spp., Gryllotalpa spp., Leucophaea maderae, Locusta spp., Periplaneta ssp., and Schistocerca spp.; from the order Isoptera, for example, Reticulitemes ssp; from the order Psocoptera, for example, Liposcelis spp.; from the order Anoplura, for example, Haematopinus spp., Linognathus spp., Pediculus spp., Pemphigus spp. and Phylloxera spp.; from the order Mallophaga, for example, Damalinea spp. and Trichodectes spp.; from the order Thysanoptera, for example, Franklinella spp., Hercinothrips spp., Taeniothrips spp., Thrips palm, Thrips tabaci and Scirtothrips auranti; from the order Heteroptera, for example, Cimex spp., Distaritiella theobroma, Dysdercus spp., Euchistus spp., Eurygaster spp., Leptocorisa spp., Nezara spp., Piesma spp., Rhodnius spp., Saldbergella singularis, Scotinophara spp., Triatoma spp., Miridae family app. such as Lygus hesperus and Lygus lineoloris, Lygaeidae family spp. such as Blissus leucopterus, and Pentatomidae family spp.; from the order Homoptera, for example, Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidun, Empoasca spp., Eriosoma larigerum, Erythroneura spp., Gascardia spp., Laodelphax spp., Lacanium corm, Lepidosaphes spp., Macrosiphus spp., Myzus spp., Nehotettix spp., Nilaparvata spp., Paratoria spp., Pemphigus spp., Planococcus spp., Pseudaulacaspis spp., Pseudococcus spp., Psylla ssp., Pulvinaria aethiopica, Quadraspidiotus app., Rhopalosiphum spp., Saissetla spp., Scaphoideus spp., Schizaphis spp., Sitobion spp., Trialeurodes vaporarlorun, Trioza erytreae and Unaspis citri; from the order Hymenoptera, for example, Acromynnex, Atta spp., Cephus spp., Diprion spp., Diprionidae, Gilpinia polytoma, Hoplocampa spp., Laslus sppp., Monomorium pharaonis, Neodiprion spp, Solenopsis spp. and Vespa ssp.; from the order Diptera, for example, Aedes spp., Antherigoria soccata, Biblo hortularius, Calliphora erythrocephala, Ceratitis spp., Chrysomyia spp., Culex spp., Cuterebra spp., Dacus spp., Drosophila melariogaster, Fannia spp., Gastrophilus spp., Glossina spp., Hypoderma spp., Hyppobosca spp., Liriomysa spp., Lucilia spp., Melanagronyza spp., Musca ssp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp., from the order Siphonaptera, for example, Ceratophyllus spp. and Xenopsylla cheopis; from the order Thysanura, for example, Lepisma saccharina; and from the order Hemiptera.

In some embodiments, the insect is a lepidopteran, coleopteran, dipteran, or hemipteran. In some embodiments, the insect is from the superfamily Aphidoidea or from the family Aleyrodidae, Chrysomelidae, Culicidae, Noctuidae, Tortricidae, Crambidae, or Erebidae. In some embodiments, the insect is from the genus Aedes, Anticarsia, Culex, Anopheles, Heliothis, Trichoplusla, Spodoptera, Chrysodeixis, Diatraea, Helicoverpa, Mythimna, Leptinotarsa, Diabrotica, or Chloridea. In some embodiments, the insect is Aedes aegpyti, Culex quinquefasciatus, Heliothis viriscens, Trichoplusia ni, Spodoptera exigua, Chrysodeixis includens, Helicoverpa zea, Spodoptera eridania, Spodoptera frugiperda, Spodoptera mauritia, Spodoptera littoralis, Spodoptera litura, Diatraea saccharalis, Diatraea grandiosella, Anopheles quadrimaculatus, Leptinotarsa decemlineata, Diabrotica barberi, Diabtrotica virgifera, Diabrotica undecimpunctata, Phyllotreta cruciferae, Cerotoma trifurcate, or Anticarsia gemmetalis.

### Methods of Making Insecticidal Compounds

Methods for synthesizing compounds described herein (e.g., a compound as provided by I, II, III, IVa-IVd, Va-Vc, or VIa-VIc, or a compound that is a substituted derivative and/or structural modification of a compound as provided by I, II, III, IVa-IVd, Va-Vc, or VIa-VIc (e.g., arundine analogs (e.g., bisindole compounds))) comprise use of chemical synthetic methods known in the art. See, e.g., Chen, J. Org. Chem. 2020, 85, 10152-10166; Mei, J. Org. Chem. 2017, 82, 7695-7707; Guo, Organic Letters 2009, 11, 4620-4623 and associated supporting information; and Kang, J. Agric. Food Chem. 2020, 68, 7839-7849, each of which is incorporated herein by reference.

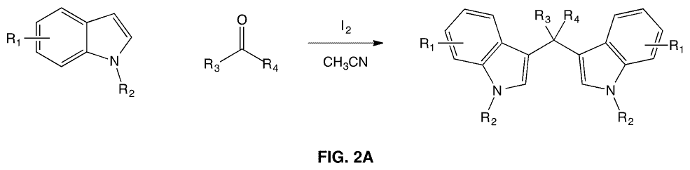

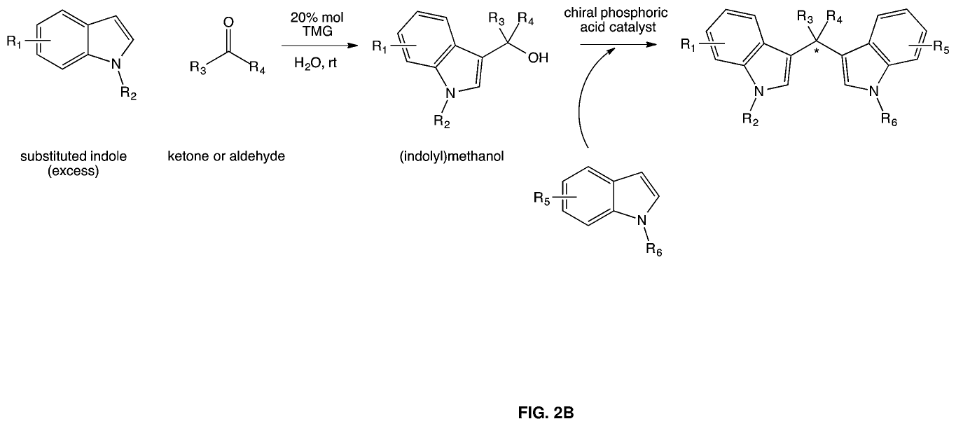

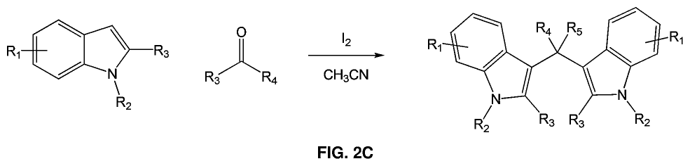

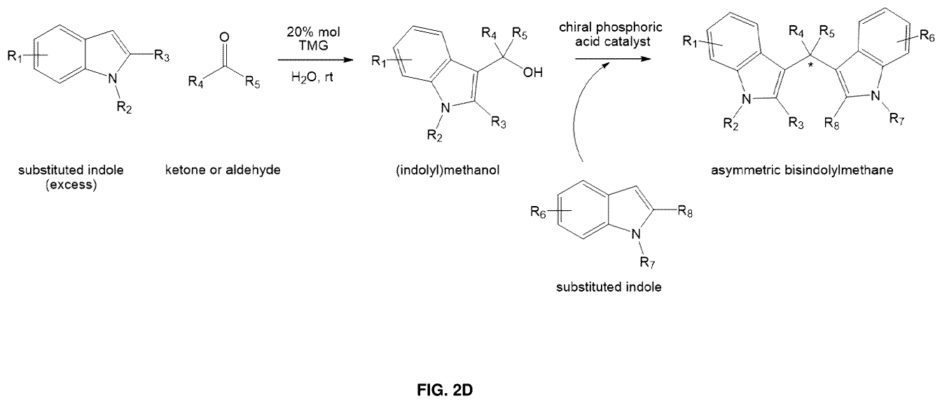

In some embodiments, the technology relates to use of bisindolylmethane as a scaffold for synthesis of insecticidal compounds. For example, embodiments of methods comprise providing bisindolylmethane as a reactant and using organic synthetic methods to modify the bisindolylmethane, e.g., to provide a bisindolylmethane derivative. Further embodiments of methods comprise reacting an indole (e.g., a substituted indole) with a ketone or aldehyde to produce a symmetrical bisindolylmethane derivative, e.g., according to the scheme shown in FIG. 2A. In some embodiments, methods comprise reacting an indole (e.g., a substituted indole) with a ketone or aldehyde to produce indolylmethanol, then using a chiral catalyst to produce an asymmetrical bisindolylmethane derivative and/or analog, e.g., according to the scheme shown in FIG. 2B.

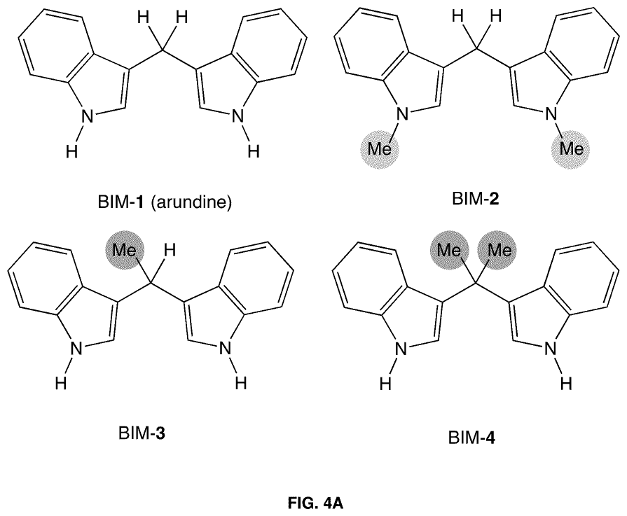

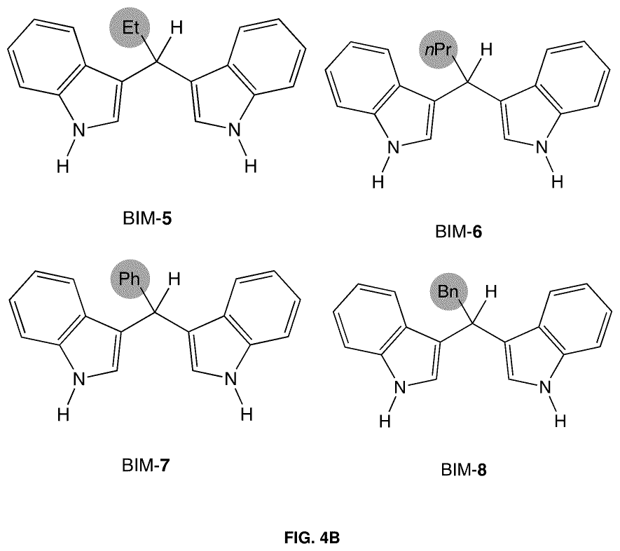

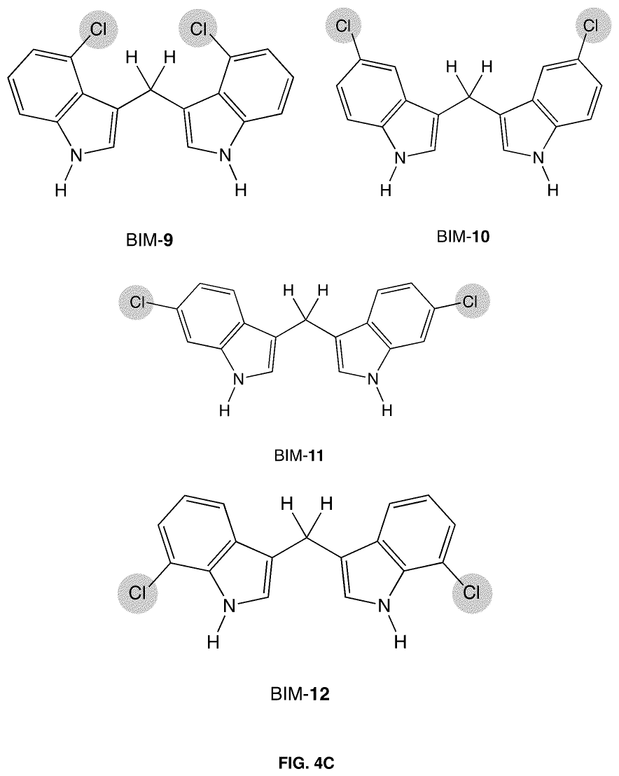

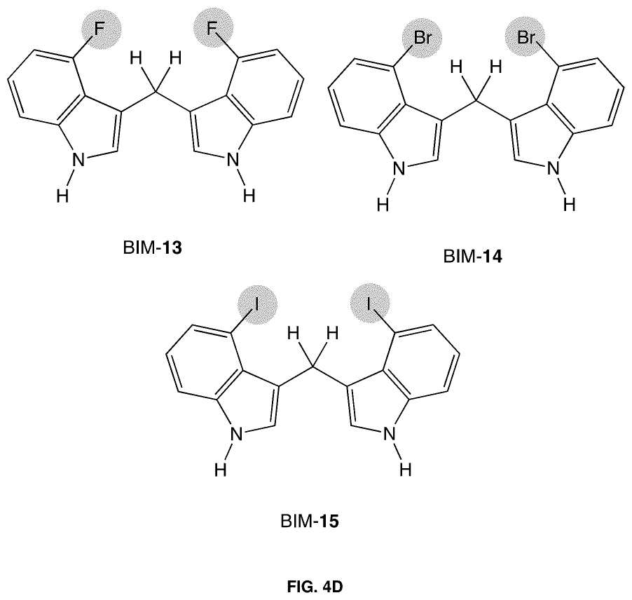

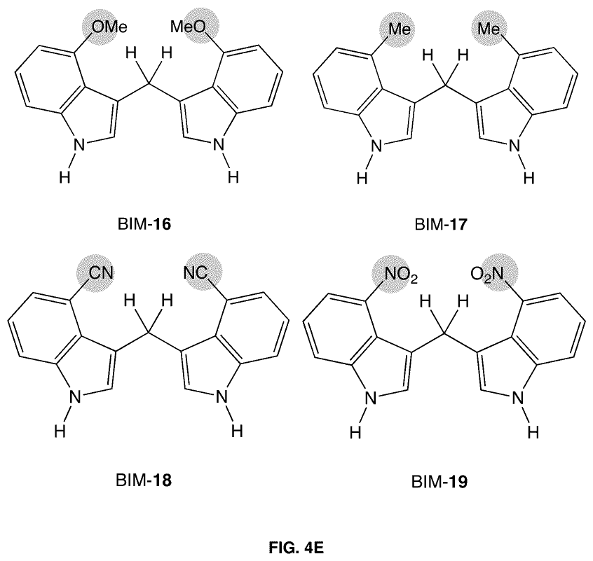

Further, symmetrical bisindolylmethane and bisheterocyclic insecticidal compounds can be prepared via one-step reaction of a substituted indole compound with an aldehyde or ketone in the presence of iodine (I₂) in a suitable polar solvent such as acetonitrile (e.g., CH₃CN) (FIG. 2C). The bisindolylmethane compounds can be purified by standard methods including normal phase and reverse phase column chromatography, preparative HPLC, size-exclusion chromatography, ion-exchange chromatography, trituration, extraction, recrystallization, and other physical separation techniques. When an acidic or basic group is present, the bisindolylmethane compounds can be formulated as various salts to improve water solubility or various formulations for biological testing. A wide variety of substituted indoles, aldehydes, and ketones are commercially available and hence bisindolylmethanes can be prepared as desired from these commercially available precursors (FIG. 4A-4F). Asymmetric bisindolymethane compounds can be obtained using a 2-step process where a substituted indole compound is first reacted with an aldehyde or ketone in the presence of 20% aqueous tetramethylguanidine (TMG) to form the indolylmethanol intermediate. Subsequent reaction with a second substituted indole compound in the presence of a chiral phosphoric acid catalyst, such as BINOL-derived phosphoric acids, can provide the desired asymmetric bisindolylmethane compounds with high enantiomeric selectivity (FIG. 2D). The asymmetric bisindolylmethane derivatives can be purified in the same manner as the symmetric bisindolylmethane derivatives.

### Methods for Making Compositions

In some embodiments, the compositions are prepared by any appropriate manufacturing processes and using any appropriate manufacturing equipment such as is known in the art. The compositions can be prepared by combining the various components in an appropriate vessel (considering vessel size, amount of composition to be made and reactivity of components) with mixing (e.g., stirring) until a uniform or homogeneous composition is achieved. The various composition components can be added sequentially, with stirring between each addition to ensure dissolution and/or dispersion of the previous component.

Although the disclosure herein refers to certain illustrated embodiments, it is to be understood that these embodiments are presented by way of example and not by way of limitation.

### EXAMPLES

### Example 1

Aedes aegypti Killing Assays

During the development of embodiments of the technology provided herein, experiments were conducted to measure the insect killing potency of compounds against Aedes aegypti larvae.

Compounds tested. 3((1H-Indol-2-yl)methyl)-1H-indole (CAS number 114648-66-7) was purchased from Ambeed (catalog #A735658); 3,3′-(pyridin-4-ylmethylene)bis(1H-indole) (CAS number 21182-09-2) was purchased from TRC Canada (catalog #P841395); indole (CAS number 120-72-9) was purchased from MilliporeSigma (catalog #442619); indole-3-carbinol (CAS number 700-06-1) was purchased from Cayman Chemical (catalog #11325); DIM-C-pPhOCH3 (CAS number 33985-68-1) was purchased from MilliporeSigma (catalog #D7946-25MG); 5-hydroxyindole (CAS number 1953-54-4) was purchased from Cayman Chemical (catalog #36431); gramine (CAS number 87-52-5) was purchased from Cayman Chemical (catalog #23401); 3,3′-diindolyl (CAS number 13637-37-1) was purchased from Carbosynth (catalog #FD66516); C-DIM12 (CAS number 178946-89-9) was purchased from MilliporeSigma (catalog #SML1508-25MG); 3,3′-methylenebis(5-fluoro-1H-indole) (CAS number 215997-93-6) was purchased from Glixx Laboratories (catalog #GLXC-20838); 3-ethyl-1H-indole (CAS number 1484-19-1) was purchased from BLD Pharm (catalog #BD86689); 3,3′-(ethane-1,1-diyl)bis(1H-indole) (CAS number 5030-91-1) was purchased from BLD Pharm (catalog #BD517796); 2-methindole (CAS number 95-20-5) was purchased from MilliporeSigma (catalog #41069); and 1,3-dimethylindole (CAS number 875-30-9) was purchased from MilliporeSigma (catalog #S705845).

Insect killing assays. Each compound was dissolved in dimethyl sulfoxide (DMSO). Killing dose curves were constructed for each compound by producing a two-fold dilution series for each compound (e.g., comprising approximately 20 serial dilutions of a stock). Dilutions were made in sterile nutrient yeast extract salt medium (NYSM) broth. The NYSM contained 8 g/L nutrient broth powder, 0.5 g/L yeast extract, 1 mM MgCl₂, 700 μM CaCl₂, and 50 μM MnCl₂ in water. The two-fold dilution series were tested in a killing bioassay as described below. Data were collected to construct a dose curve and determine the half-maximal lethal concentration (LC₅₀) value.

Bioassays were performed using second or third instar Aedes aegypti larvae, with biological replication. Ae. aegpyti larvae were purchased from Benzon Research, Inc. (Carlisle, PA). Larvae were washed with sterile water before transfer to 24-well assay plates containing 1 mL of sterile water in each well. Each well contained 5 larvae. The final DMSO concentration in each assay well was 1%. Larval mortality was scored for each well at 24-hour timepoints after application. LC₅₀ values were determined using mortalities collected at 72 hours (Table 1). In Table 1, mean mortalities and standard deviations were calculated from at least three independent biological replicates. 3,3′-diindolyl did not display any bioactivity against Aedes aegypti. LC₅₀ values were calculated using GraphPad Prism software.

TABLE 1
killing potency against Aedes aegypti

| | | Molecular weight | LC₅₀ | LC₅₀ |
|---|---|---|---|---|
| Compound Name | CAS Number | (g/mol) | (μg/mL) | (μM) |
| 3-((1H-indol-2-yl)methyl)-1H- | 114648-66-7 | 246.31 | 5.5 ± 2.4 | 22.5 ± 9.9 |
| indole | | | | |
| 3,3′-(pyridin-4- | 21182-09-2 | 323.4 | 62.9 ± 6.8 | 194.4 ± 21 |
| ylmethylene)bis(1H-indole) | | | | |
| indole | 120-72-9 | 117.15 | 40.2 ± 6.9 | 343.5 ± 59.2 |
| indole-3-carbinol | 700-06-1 | 147.17 | 66.7 ± 45.2 | 453.2 ± 307 |
| 1,1-bis(3′-indolyl)-1-(p- | 33985-68-1 | 352.43 | 23.4 ± 19.8 | 66.6 ± 56.4 |
| methoxyphenyl)methane | | | | |
| 5-hydroxyindole | 1953-54-4 | 133.15 | 278.6 ± 25.9 | 2092.6 ± 194 |
| gramine | 87-52-5 | 174.24 | 188.7 ± 55.1 | 1083 ± 316 |
| 3,3′-diindolyl | 13637-37-1 | 232.28 | >2500 | >1000 |
| 3,3′-((4- | 178946-89-9 | 356.8 | 2.6 ± 0.2 | 7.3 ± 0.6 |
| chlorophenyl)methylene)bis(1H- | | | | |
| indole) | | | | |
| 3,3′-methylenebis(5-fluoro-1H- | 215997-93-6 | 282.29 | 2.2 ± 0.9 | 7.8 ± 3.2 |
| indole) | | | | |
| 3-ethyl-1H-indole | 1484-19-1 | 145.2 | 8.7 ± 3.5 | 60 ± 24.2 |
| 3,3′-(ethane-1,1-diyl)bis(1H- | 5030-91-1 | 260.3 | 4.2 ± 1.6 | 16.1 ± 6.1 |
| indole) | | | | |
| 2-methindole | 95-20-5 | 131.17 | 6.8 ± 0.5 | 52.2 ± 3.6 |
| 1,3 dimethylindole | 875-30-9 | 145.2 | 13.8 ± 3.0 | 95.2 ± 21 |

As shown in Table 1, certain commercially available indole and bisindole compounds display killing bioactivity against Aedes aegypti.

### Example 2

Spodoptera frugiperda Killing Assays

During the development of embodiments of the technology provided herein, experiments were conducted to measure the insect killing potency of compounds against Spodoptera frugiperda (fall armyworm) larvae.

Compounds tested. 3,3′-Diindolylmethane (also called arundine, CAS number: 1968-05-4) was purchased from MilliporeSigma (catalog #74601); 3,3′-diindolyl (CAS number 13637-37-1) was purchased from Carbosynth (catalog #FD66516); 3,3′-(pyridin-4-ylmethylene)bis(1H-indole) (CAS number 21182-09-2) was purchased from TRC Canada (catalog #P841395); and 3((1H-Indol-2-yl)methyl)-1H-indole (CAS number 114648-66-7) was purchased from Ambeed (catalog #A735658). Additional BIM compounds were synthesized using the reagents listed in Table 3 according to the reaction schemes provided in Example 3. These compounds were also tested for killing potency according to the methods described below.

Insect killing assays. Each compound was dissolved in dimethyl sulfoxide (DMSO). Killing dose curves were constructed for each compound by producing a two-fold dilution series of the compound (e.g., comprising approximately 16 serial dilutions of a stock for 3,3′-diindolylmethane and 3-((1H-Indol-2-yl)methyl)-1H-indole and comprising approximately 12 serial dilutions of a stock for 3,3′-diindolyl and 3,3′-(pyridin-4-ylmethylene)bis(1H-indole)). Dilutions were made in sterile NYSM broth as described above. The two-fold dilution series were tested in a killing bioassay as described below. Data were collected to construct a dose curve and determine the half-maximal lethal concentration (LC₅₀) value.

Bioassays were performed using first instar larvae of the Lepidopteran species Spodoptera frugiperda (fall armyworm), with an N=16 for each concentration of compound tested. S. frugiperda larvae were purchased from Benzon Research, Inc. (Carlisle, PA). A diet-surface overlay format was used in which 1 milliliter of standard lepidopteran diet was placed in each well. One hundred microliters of sample were pipetted into each well and allowed to dry. A DMSO control was included with each set of samples. Spinosad (MilliporeSigma, catalog #33706) was used as a positive control for insecticidal activity, and water was used as an additional negative control.

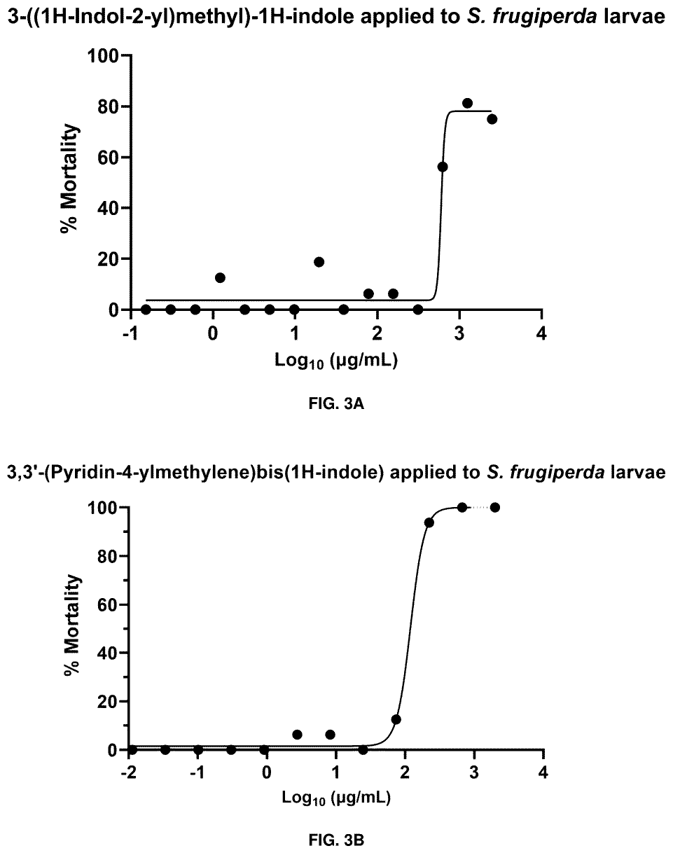

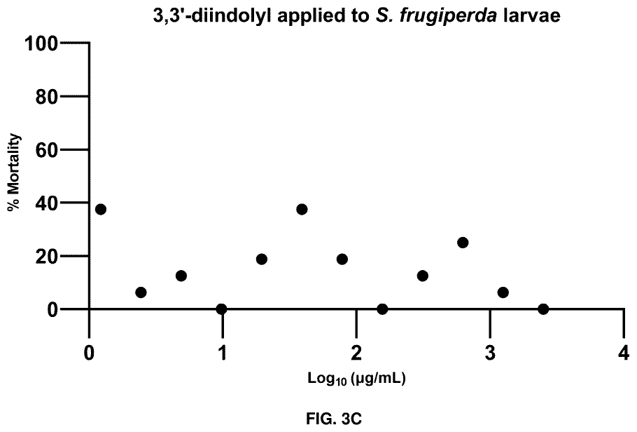

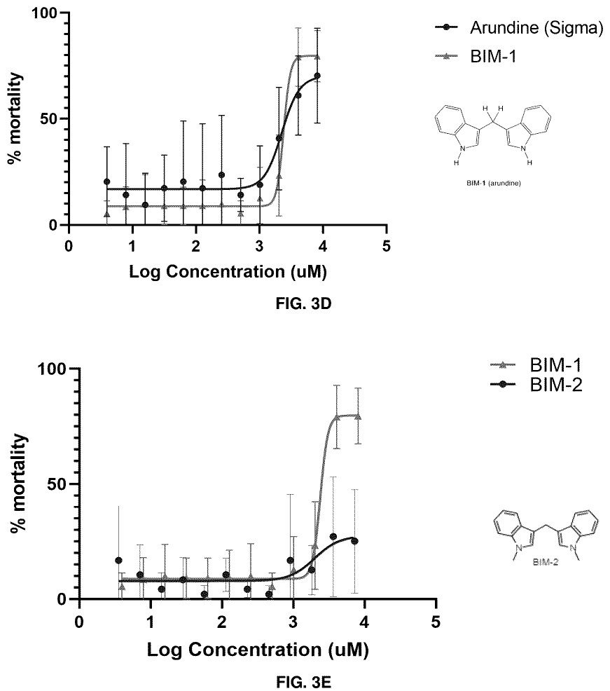

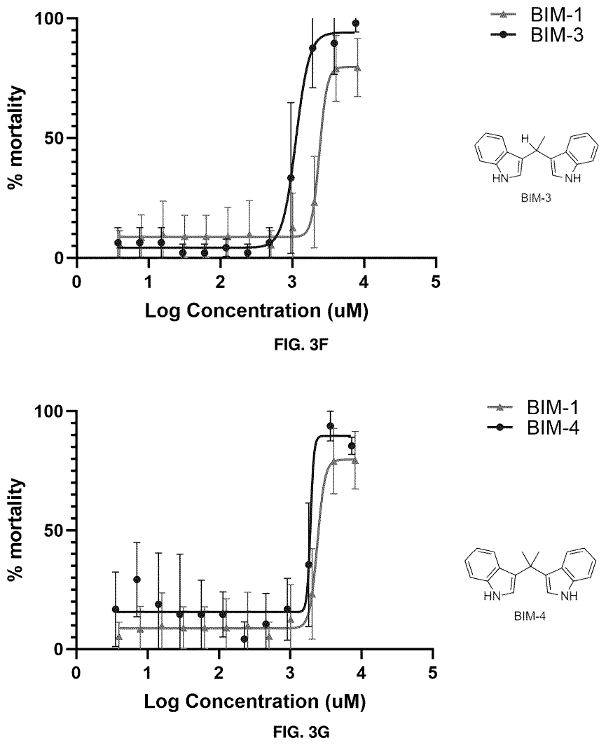

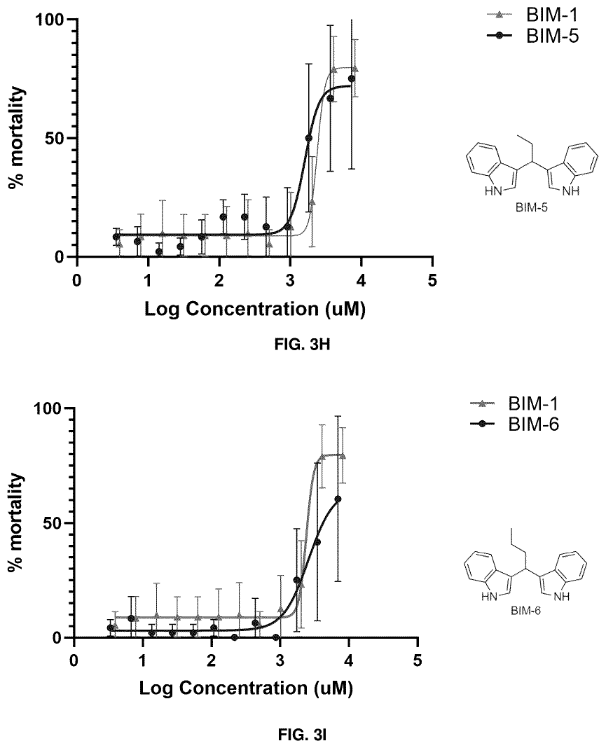

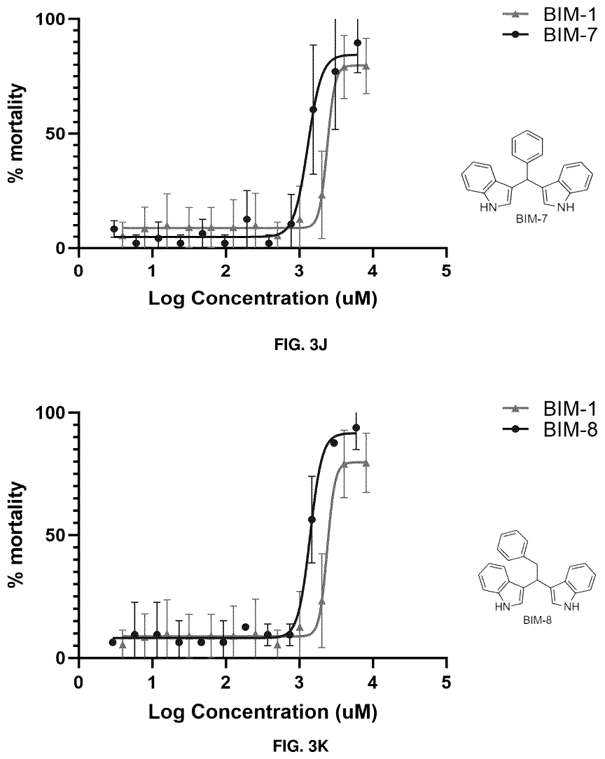

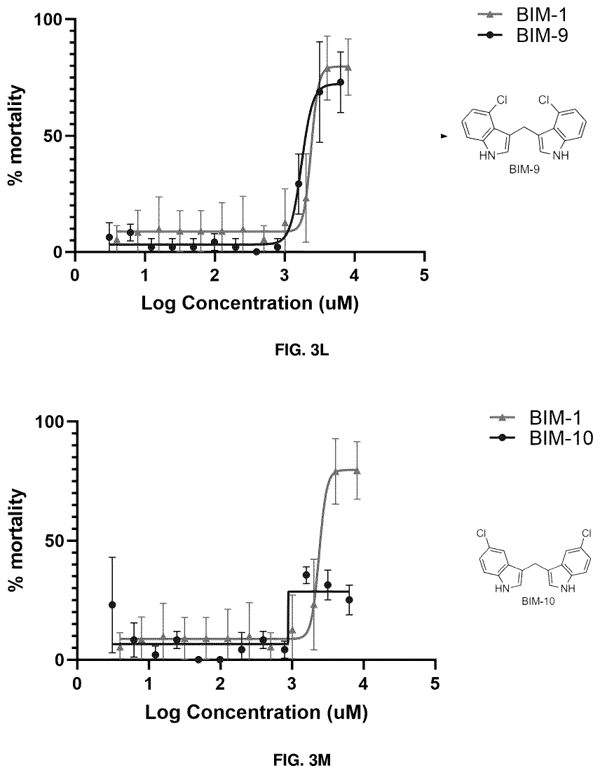

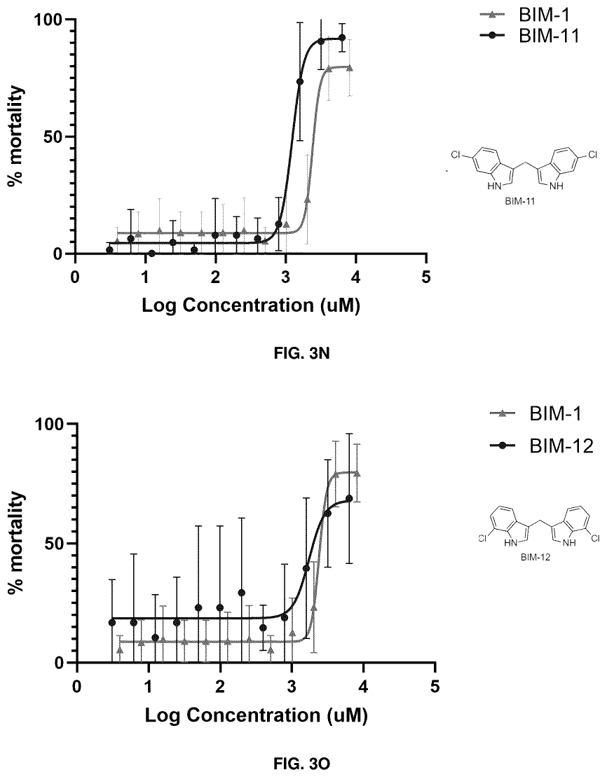

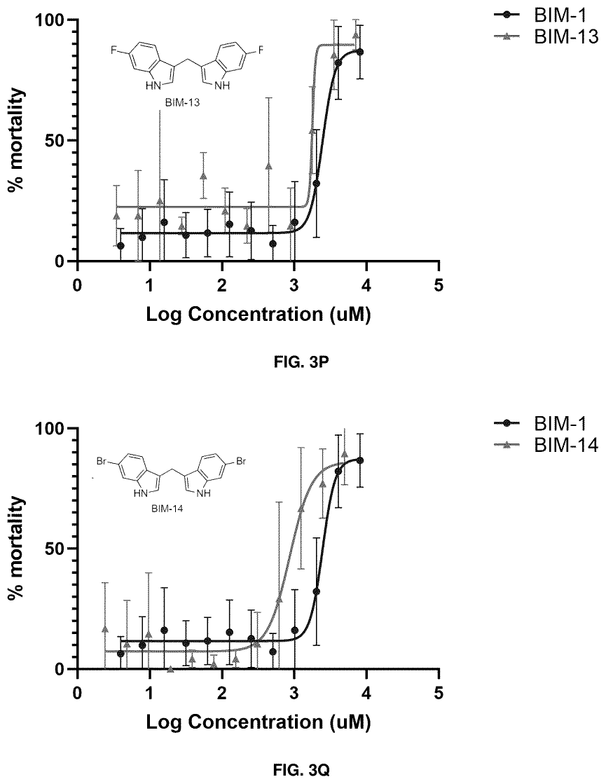

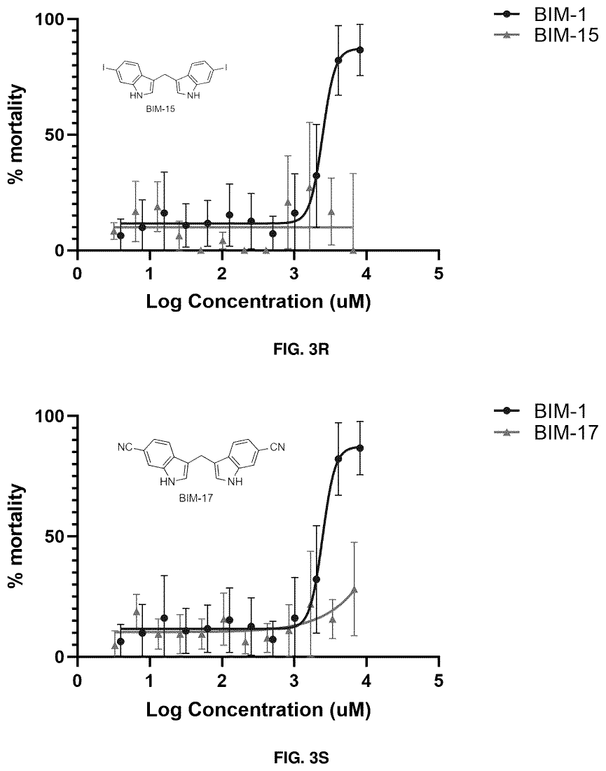

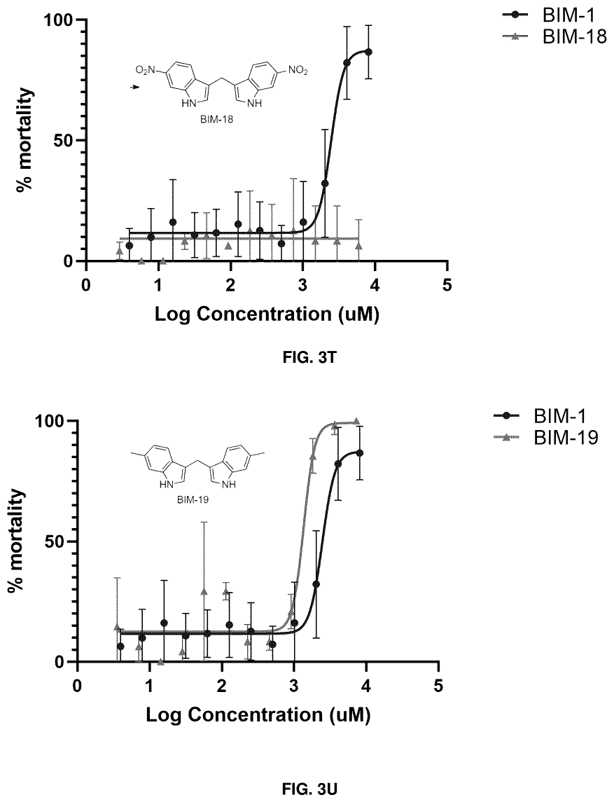

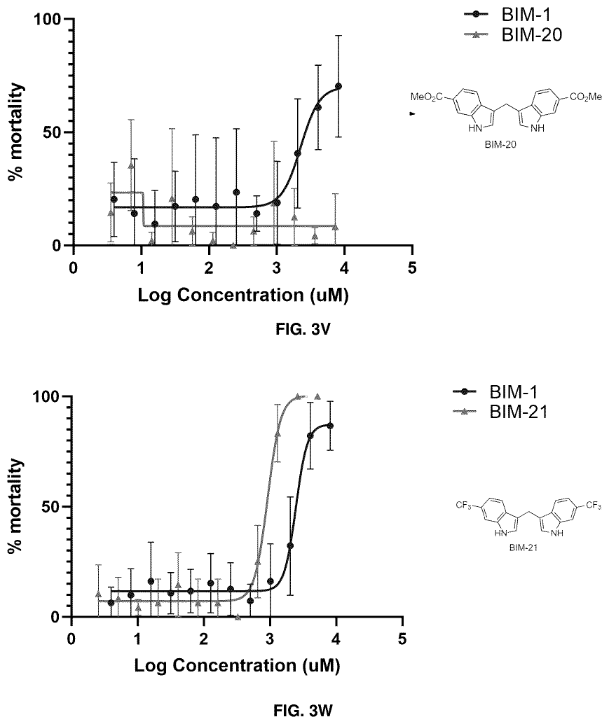

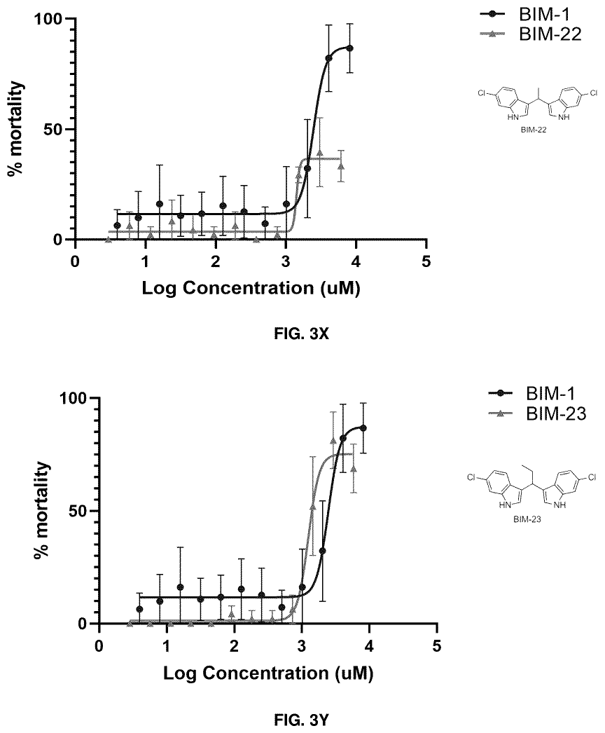

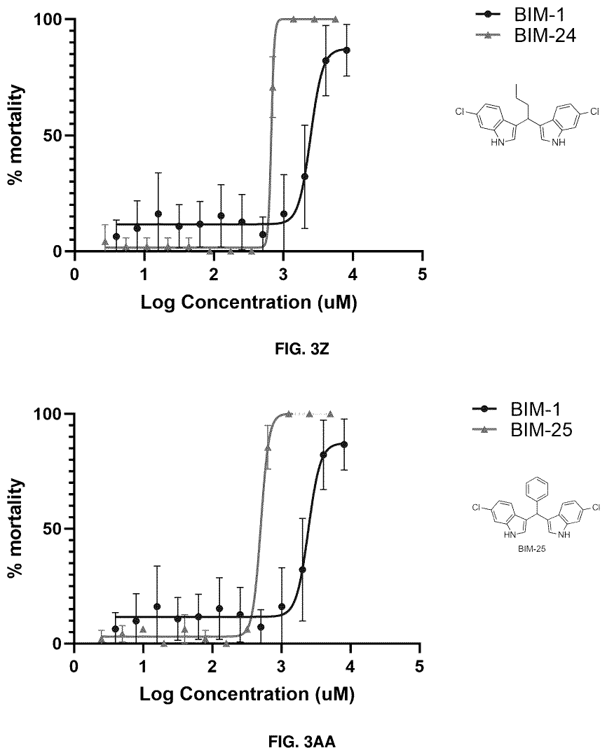

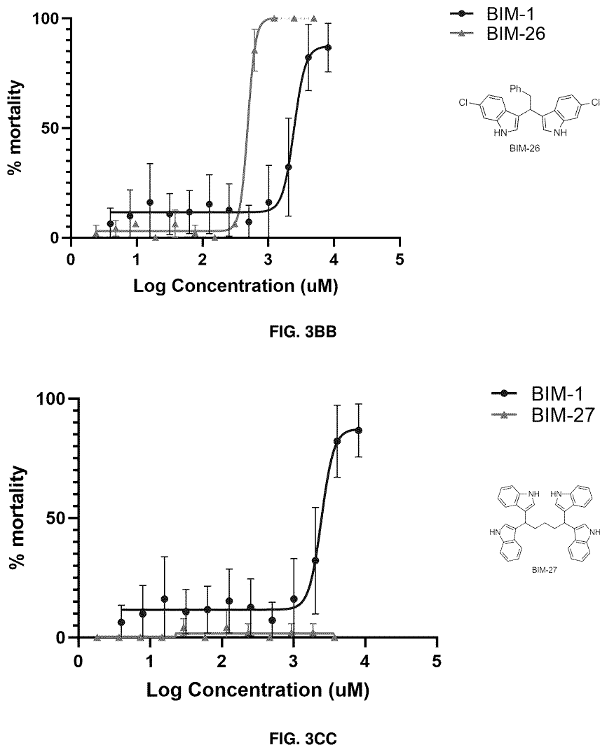

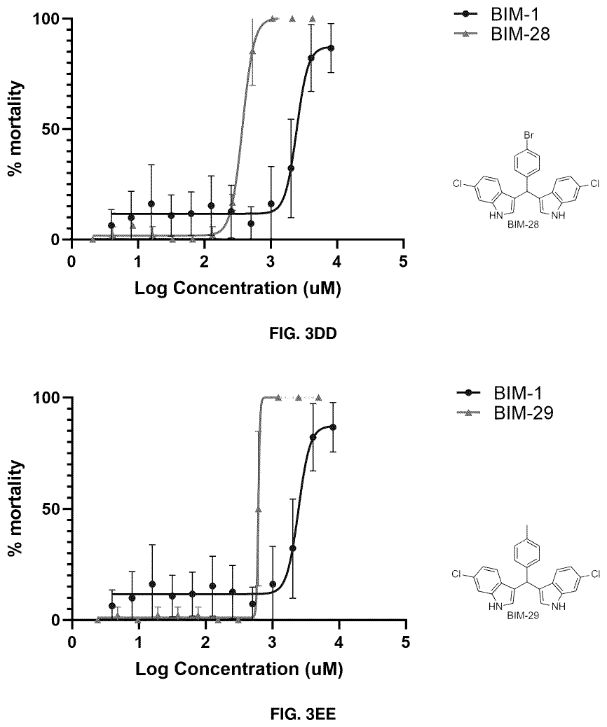

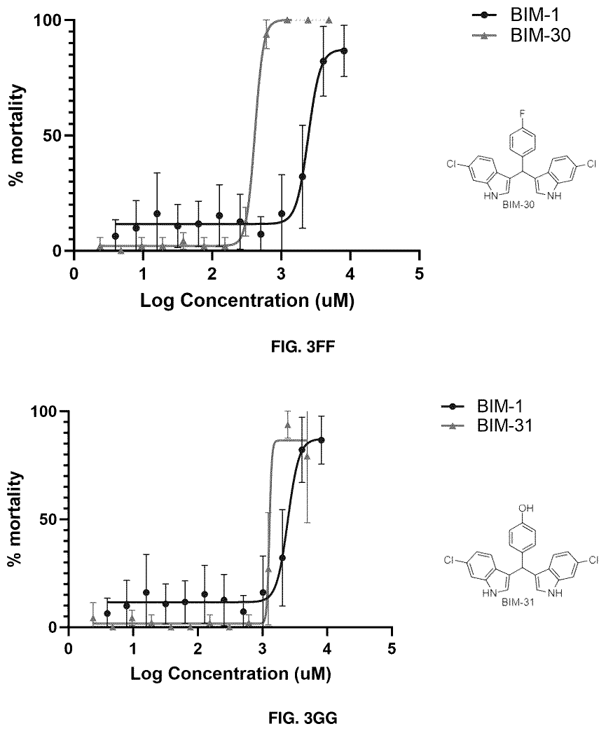

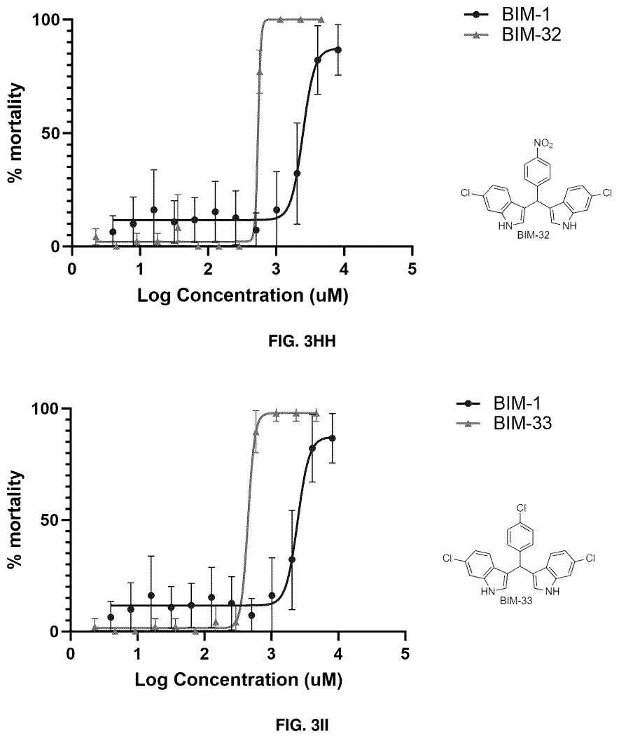

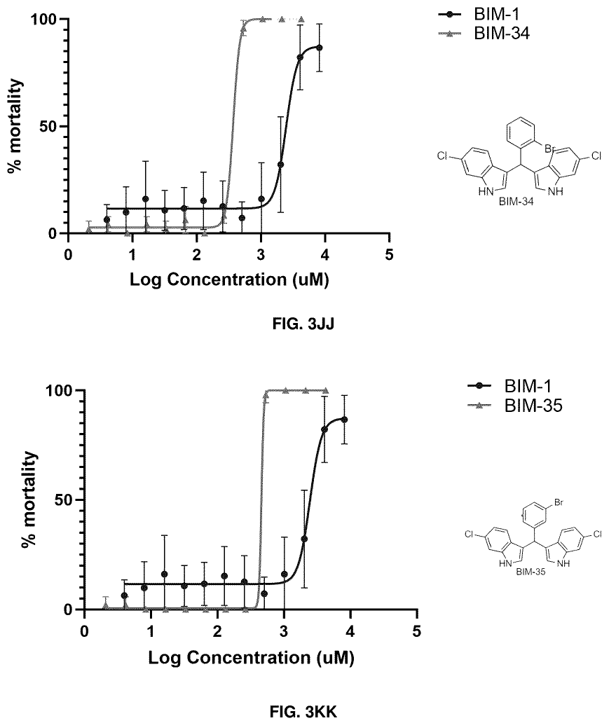

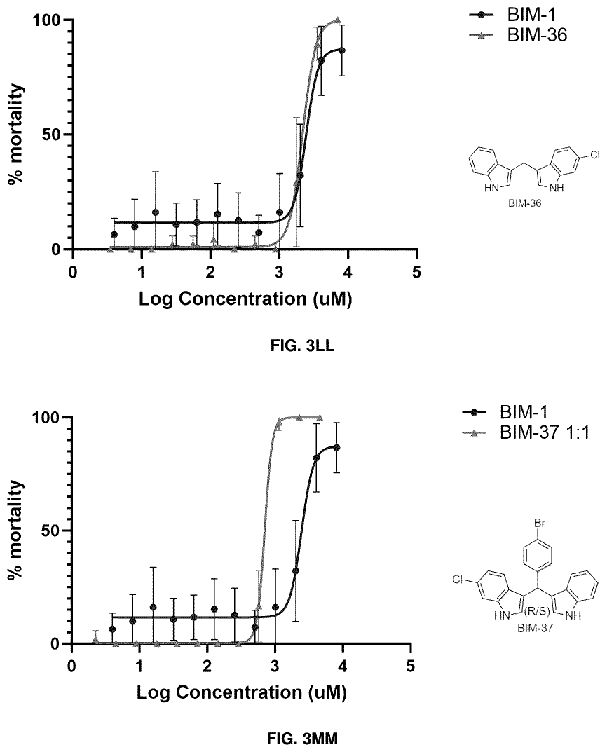

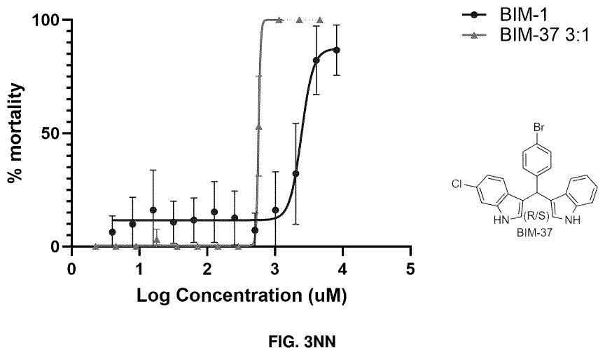

As indicated by the dose curve shown in FIG. 3A, the bisindole compound 3-((1H-indol-2-yl)methyl)-1H-indole is active against S. frugiperda. Mortality data collected from the killing bioassay described above are plotted against log₁₀-transformed compound concentrations (N=16 larvae for each concentration). An inhibitory dose-response curve was fitted using GraphPad Prism software (R²=0.96). The representative curve fit shown corresponds to a 3((1H-indol-2-yl)methyl)-1H-indole LC₅₀ of 598.2 μg/mL (2.4 mM). Mortality in the negative control was not subtracted but was less than 10% for all assays.

Further, as indicated by the dose curve shown in FIG. 3B, the bisindole compound 3,3′-(pyridin-4-ylmethylene)bis(1H-indole) is active against S. frugiperda. Mortality data collected from the killing bioassay described above are plotted against log₁₀-transformed compound concentrations (N=16 larvae for each concentration). An inhibitory dose-response curve was fitted using GraphPad Prism software (R²=0.99). The representative curve fit shown corresponds to a 3,3′-(pyridin-4-ylmethylene)bis(1H-indole) LC₅₀ of 119.6 μg/mL (369.8 μM). Mortality in the negative control was not subtracted but was less than 10% for all assays.

As indicated by the data shown in FIG. 3C, the bisindole compound 3,3′-diindolyl is not active against S. frugiperda. Mortalities of S. frugiperda larvae subjected to a range of 3,3′-diindolyl concentrations were collected. Mortality data collected from the killing bioassay described above are plotted against log₁₀-transformed compound concentrations (N=16 larvae for each concentration). An attempt to fit an inhibitory dose-response curve to the data using GraphPad Prism software did not result in a satisfactory fit (R²<0.5). The data collected indicate that 3,3-diindolyl has an LC₅₀ of greater than 2500 μg/mL (greater than 10 mM). Mortality in the negative control was not subtracted but was less than 10% for the assay.

During the development of embodiments of the technology described herein, data were collected indicating that certain bisindole compounds had killing activity against insects (e.g., dipterans and lepidopterans). The bisindole compound 3-((1H-indol-2-yl)methyl)-1H-indole is active against the lepidopteran S. frugiperda (FIG. 3A). This compound was previously shown to be active against the dipteran Aedes aegypti. See, e.g., U.S. patent application Ser. No. 17/724,078, which is incorporated by reference herein in its entirety. Further, Example 1 and Example 2, respectively, provide data indicating that the bisindole compound 3,3′-(pyridin-4-ylmethylene)bis(1H-indole) is active against both Ae. aegypti and S. frugiperda (Table 1 and FIG. 3B). Finally, data provided in Example 1 and Example 2 indicate that not all bisindole compounds have insect killing activity. In particular, the bisindole compound 3,3′-diindolyl does not have significant killing activity toward Ae. aegypti or S. frugiperda (Table 1 and FIG. 3C).

FIG. 3D to FIG. 3NN are killing dose curves for BIM compounds (e.g., as synthesized and characterized below) against first instar larvae of S. frugiperda (fall armyworm). Table 2 summarizes the LD₅₀ values calculated from the killing dose curves of FIG. 3D to FIG. 3NN for the BIM compounds tested in S. frugiperda killing assays. The compounds are indicated by a “BIM” number and are listed in Example 3.

TABLE 2
LD₅₀ for BIM compounds

| Compound | | LD₅₀ | Standard deviation |
|---|---|---|---|
| Series | BIM | (μM) | (μM) |
| | Arundine | 1825 | 425 |
| 1 | BIM-1 | 2478 | 615 |
| 1 | BIM-2 | >3000 | |
| 1 | BIM-3 | 1277 | 374 |
| 1 | BIM-4 | 1814 | 59 |
| 2 | BIM-5 | 2379 | 653 |
| 2 | BIM-6 | 3235 | 184 |
| 2 | BIM-7 | 1683 | 534 |
| 2 | BIM-8 | 1477 | 64 |
| 3 | BIM-9 | 1564 | 290 |
| 3 | BIM-10 | >3000 | |
| 3 | BIM-11 | 1185 | 369 |
| 3 | BIM-12 | 1175 | 264 |
| 4 | BIM-13 | 1849 | 138 |
| 4 | BIM-14 | 934.3 | 366 |
| 4 | BIM-15 | >10000 | |
| 4 | BIM-17 | >10000 | |
| 4 | BIM-18 | >10000 | |
| 4 | BIM-19 | 1406 | 244 |
| 4 | BIM-20 | >3000 | |
| 4 | BIM-21 | 974 | 372 |
| 5 | BIM-22 | <3000 | |
| 5 | BIM-23 | 1346 | 79 |
| 5 | BIM-24 | 678 | 16 |
| 5 | BIM-25 | 504 | 45 |
| 5 | BIM-26 | 581 | 42 |
| 6 | BIM-27 | >5000 | |
| 6 | BIM-28 | 371 | 61 |
| 6 | BIM-29 | 611 | 41 |
| 6 | BIM-30 | 406 | 56 |
| 6 | BIM-31 | 1417 | 235 |
| 6 | BIM-31 | 542 | 16 |
| 6 | BIM-33 | 485 | 75 |
| 6 | BIM-34 | 352 | 36 |
| 6 | BIM-35 | 429 | 40 |
| 7 | BIM-36 | 2457 | 811 |
| 7 | BIM-37 R/S 1:1 | 725 | 138 |
| 7 | BIM-37 R/S 3:1 | 576 | 21 |

### Example 3

### Bisindole Synthetic Series

Based on the structure-activity relationships indicated by the data collected in Example 1 and in Example 2 using commercially available compounds, experiments were conducted to synthesize additional arundine analogs (e.g., bisindole compounds) and test their killing activities.

First, the methyl substituted arundine derivatives of Series 1 were synthesized (FIG. 4A) to evaluate if adding a substituent to the indole amine is beneficial and to evaluate if monosubstitution or disubstitution of the carbon in the linker joining the two indole rings is beneficial. Substituents that improve activity were carried forward in subsequent series. The methyl group on the indole ring was explored further in Series 5.

Further, some of the arundine substituted derivatives of Series 2-5 (FIG. 4B to FIG. 4E) were synthesized to identify optimal identity and placement of substituents on the linker and indole rings. The arundine substituted derivatives of Series 2 were produced to identify the optimal size of a functional group to add to the linker (FIG. 4B). If adding substituents to the linker carbon and/or to the indole amine produce increased activity based on killing assays of Series 1 and Series 2 compounds, such compounds were also studied with respect to the identity and placement of substituents on the indole rings in Series 3-5.

The arundine substituted derivatives of Series 3 were produced to identify the optimal position for a halogen on the indoles (FIG. 4C). If adding substituents to the linker carbon and/or to the indole amine produce increased activity based on killing assays of Series 1 and Series 2 compounds, such compounds were also studied with respect to identifying an optimal position for a halogen on the indoles.

The arundine substituted derivatives of Series 4 (FIG. 4D) were produced to identify the optimal halogen substituent to add at the optimal site (ring position 4, 5, 6, or 7) for indole substitution as identified in Series 3.

The arundine substituted derivatives of Series 5 (FIG. 4E) were produced to study a strong electron donating group, a weak electron donating group, a weak electron withdrawing group, and a strong electron withdrawing group as substituents on the indole ring. The optimal site (ring position 4, 5, 6, or 7) for indole substitution were used as identified in Series 3.

Table 3 provides reagents used to synthesize the arundine substituted derivatives. Note that three variations of BIM-29 are providing having F, OH, and methyl substituents.

TABLE 3
Synthesis of arundine substituted derivatives

| Compound | Indole reagent | Linker |
|---|---|---|
| BIM-1 | Indole | Formaldehyde |
| (Arundine) | | |
| BIM-2 | 1-methylindole | Formaldehyde |
| BIM-3 | Indole | Acetaldehyde |
| BIM-4 | Indole | Acetone |
| BIM-5 | Indole | Propionaldehyde |
| BIM-6 | Indole | Butyraldehyde |
| BIM-7 | Indole | Benzaldehyde |
| BIM-8 | Indole | Phenylacetaldehyde |
| BIM-9 | 4-chroroindole | Formaldehyde |
| BIM-10 | 5-chloroindole | Formaldehyde |
| BIM-11 | 6-chloroindole | Formaldehyde |
| BIM-12 | 7-chroloindole | Formaldehyde |
| BIM-13 | 6-fluoroindole | Formaldehyde |
| BIM-14 | 6-bromoindole | Formaldehyde |
| BIM-15 | 6-iodoindole | Formaldehyde |
| BIM-16 | 4-methoxyindole | Formaldehyde |
| | 5-methoxyindole | |
| | 6-methoxyindole | |
| | 7-methoxyindole | |
| BIM-17 | 6-cyanoindole | Formaldehyde |
| BIM-18 | 6-nitroindole | Formaldehyde |
| BIM-19 | 6-methylindole | Formaldehyde |
| BIM-20 | 6-methoxyindole | Formaldehyde |
| BIM-21 | 6-trifluoromethylindole | Formaldehyde |
| BIM-23 | 6-chloroindole | Propionaldehyde |
| BIM-24 | 6-chloroindole | Butyraldehyde |
| BIM-25 | 6-chloroindole | Benzaldehyde |
| BIM-26 | 6-chloroindole | Phenylacetaldehyde |
| BIM-27 | Indole | Gluteraldehyde |
| BIM-28 | 6-chloroindole | 4-Bromobenzylaldehyde |
| BIM-29 F | 6-chloroindole | 4-Fluorobenzylaldehyde |
| BIM-29 OH | 6-chloroindole | 4-Hydroxybenzylaldehyde |
| BIM-29 Me | 6-chloroindole | 4-Methylbenzaldehyde |
| BIM-32 | 6-chloroindole | 4-Nitrobenzaldehyde |
| BIM-33 | 6-chloroindole | 4-Chlorobenzaldehyde |
| BIM-34 | 6-chloroindole | 2-Bromobenzaldehyde |
| BIM-35 | 6-chloroindole | 3-Bromobenzaldehyde |

Asymmetric BIMs

| | | |
|---|---|---|
| BIM-36 | 1-H-indoly-3-yl methanol | 6-chloroindole |
| BIM-37 R/S 1:1 | 6-chloroindole, Indole | 4-bromobenzaldehyde |
| BIM-37 R/S 3:1 | 6-chloroindole, Indole | 4-bromobenzaldehyde |

Synthesis schemes for the BIM compounds and characterization of products are provided below:

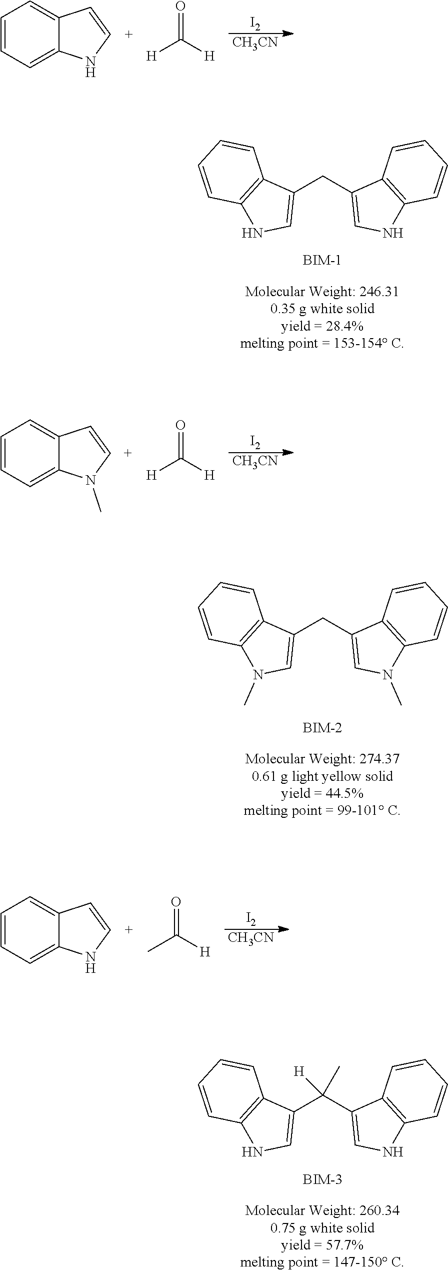

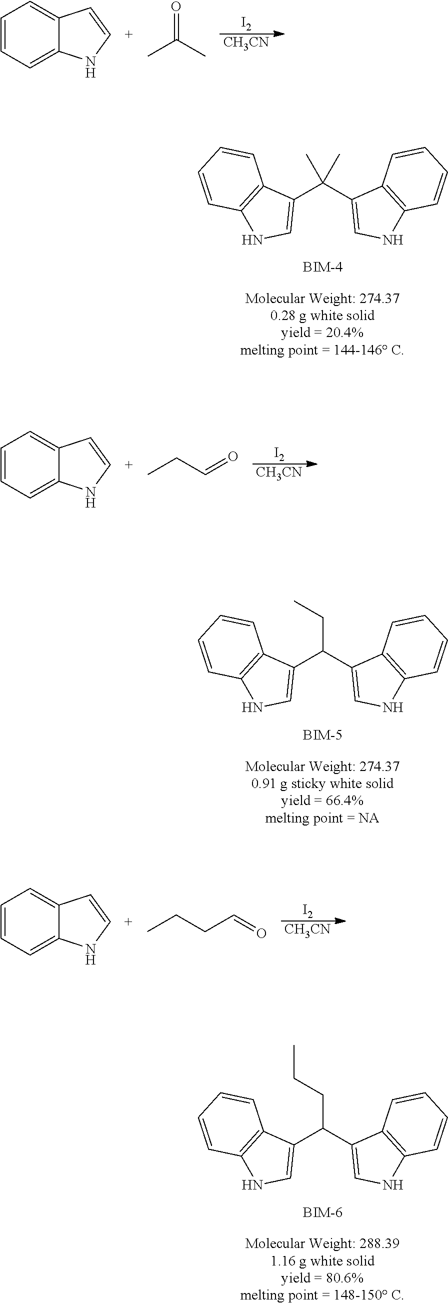

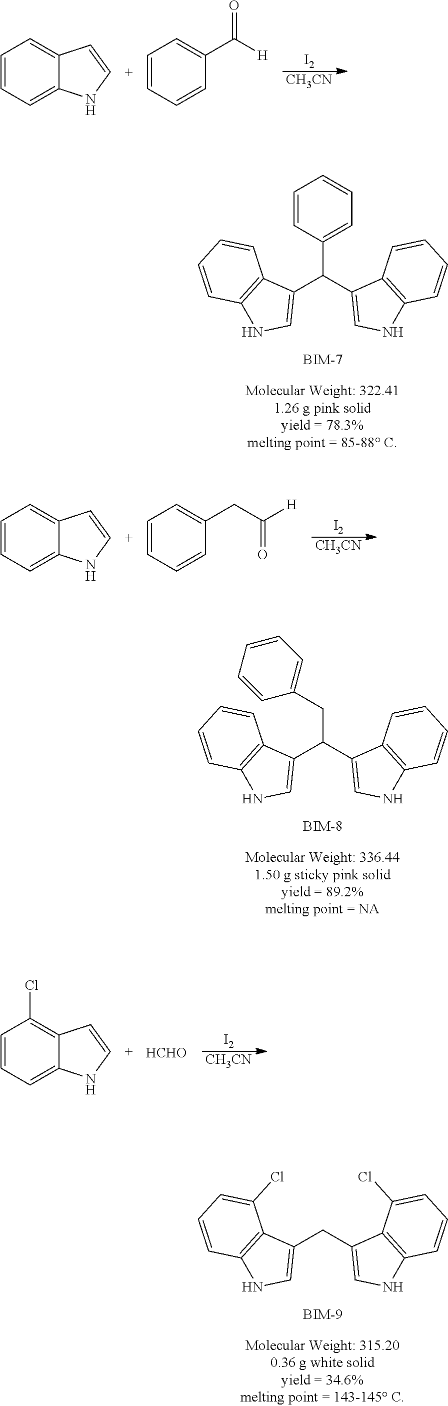

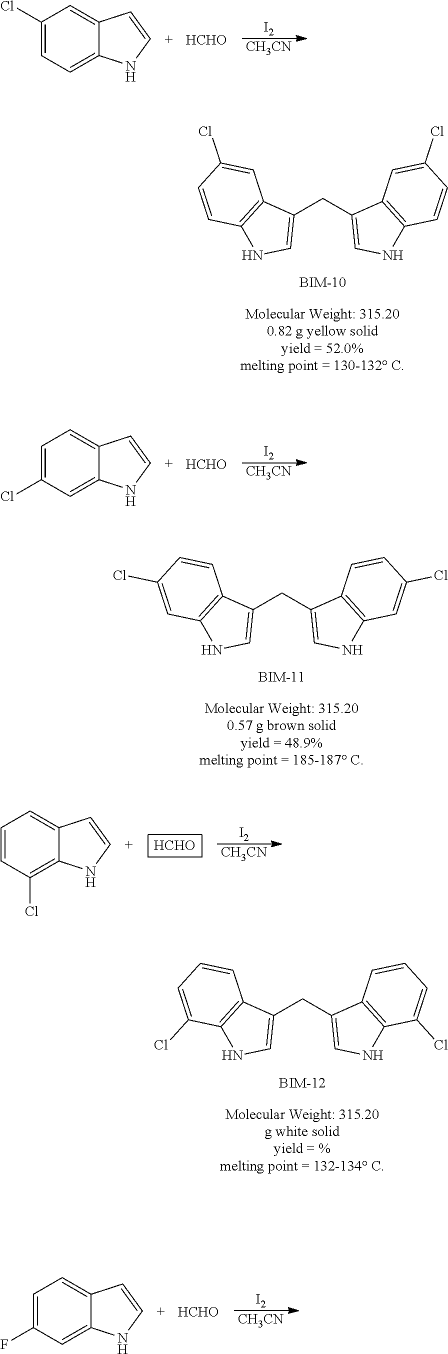

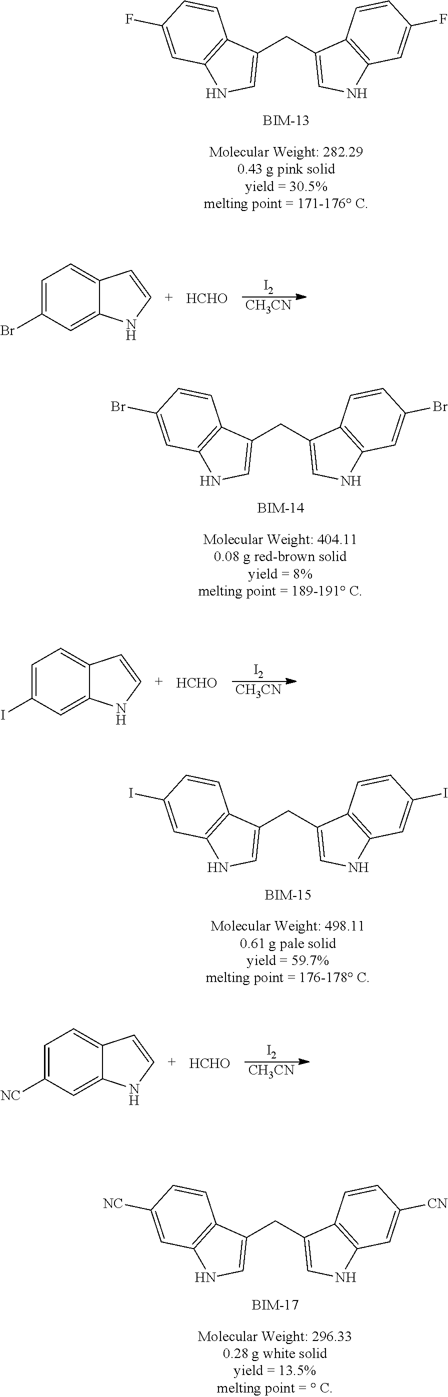

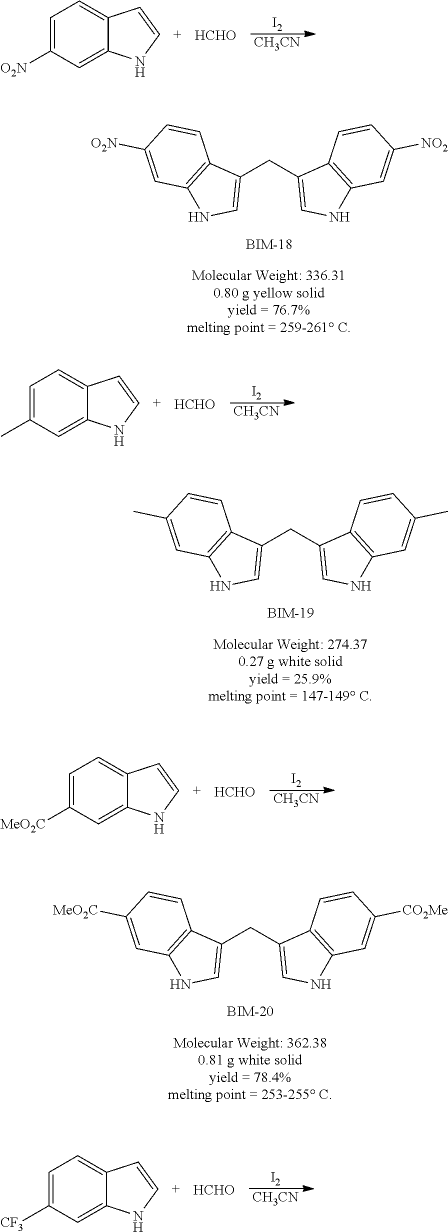

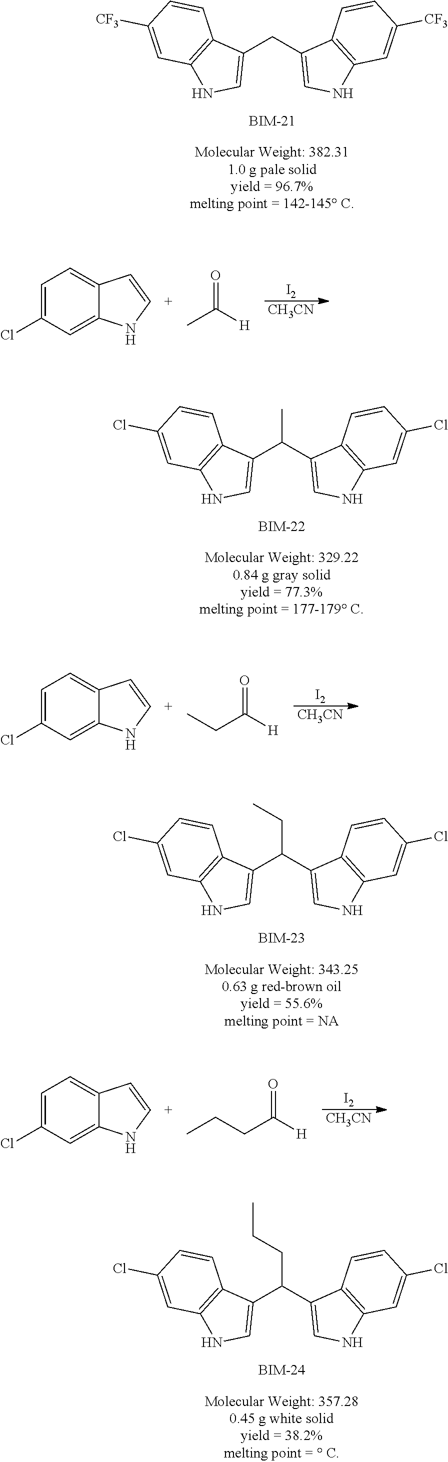

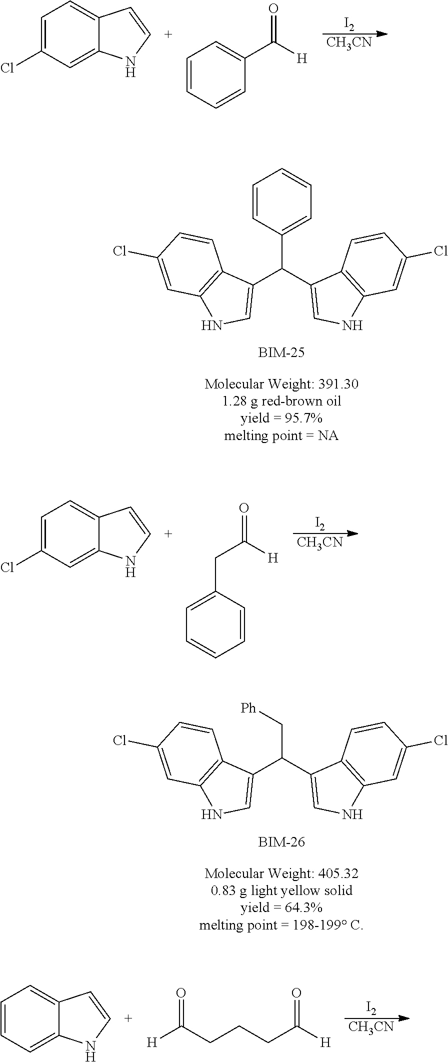

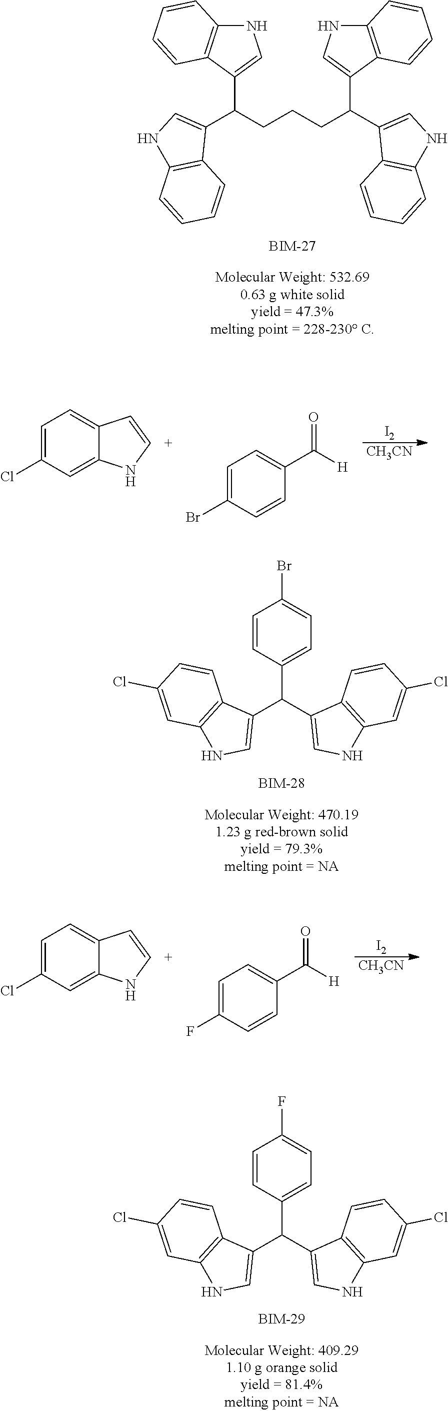

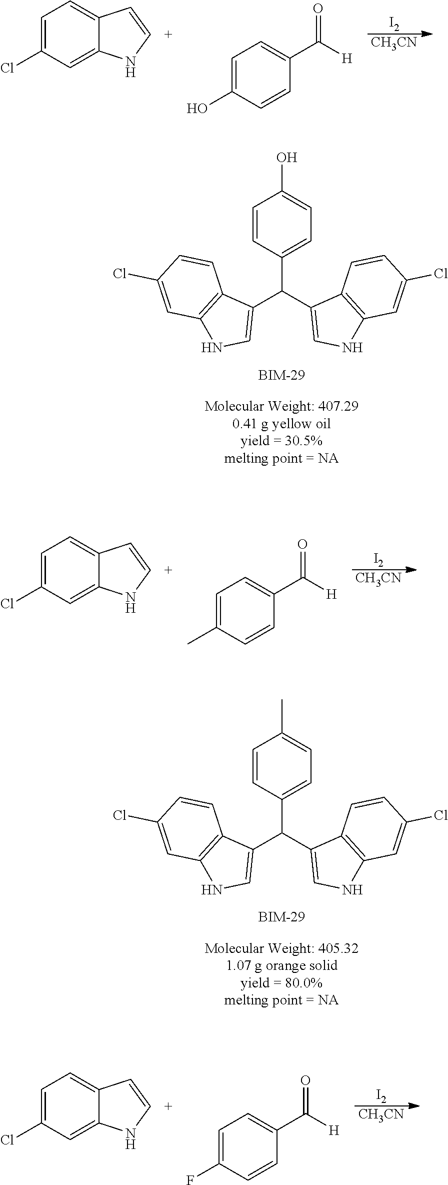

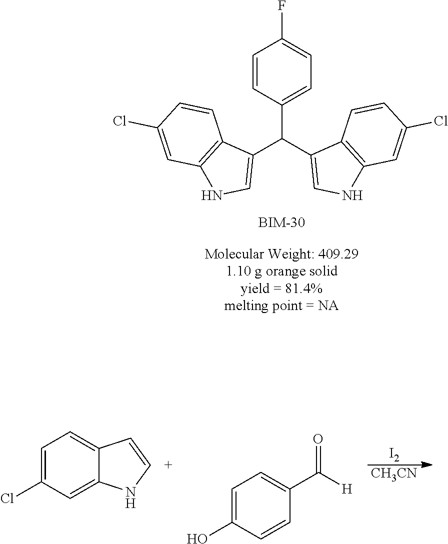

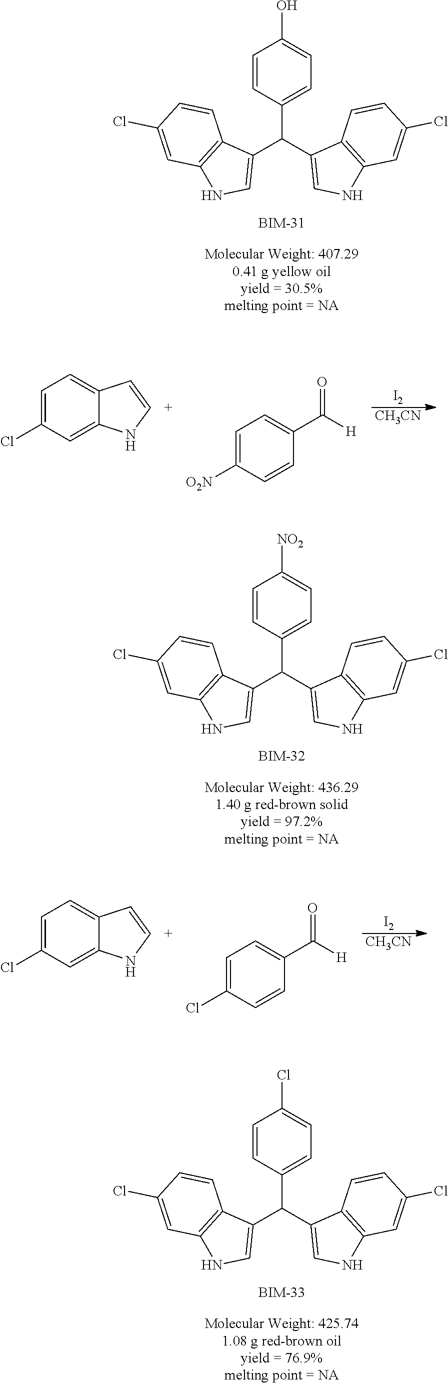

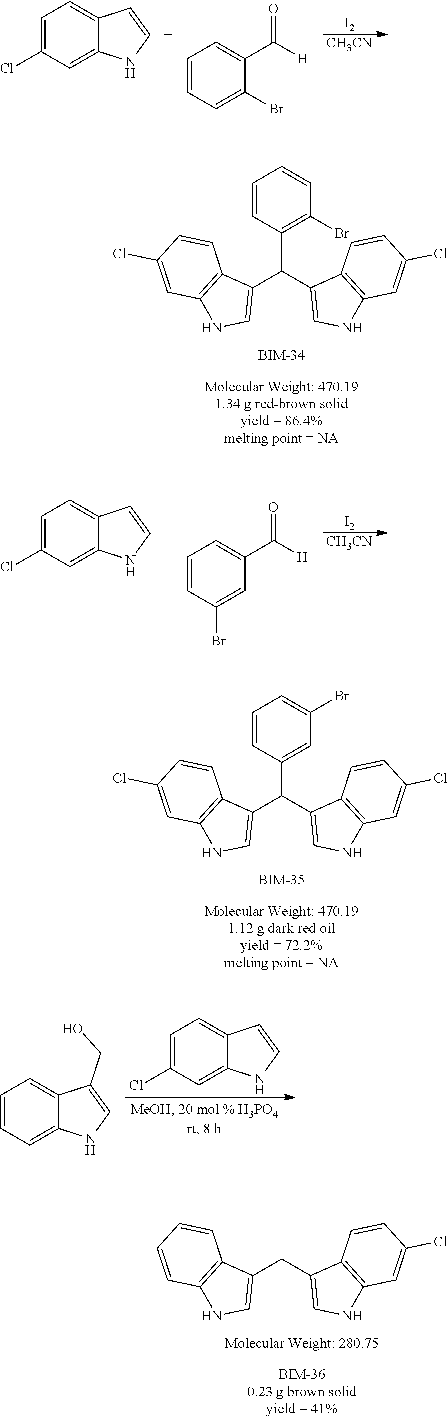

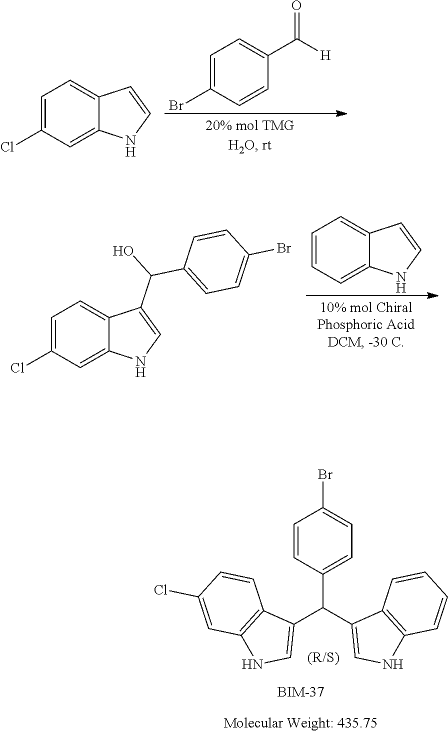

### Example 4

### Testing Killing of BIM Compounds Against Additional Insect Species

During the development of embodiments of the technology described herein, experiments were conducted to test the killing of arundine against a panel of 11 insect species. Killing assays were performed as described in Example 2 for Spodoptera frugiperda. Briefly, arundine was dissolved in dimethyl sulfoxide (DMSO). Killing dose curves were constructed by producing a two-fold dilution series of arundine. Dilutions were made in sterile NYSM as described. The two-fold dilution series were tested in a killing bioassay performed using first instar larvae of the species listed in Table 4, with an N=16 for each concentration of compound tested. A diet-surface overlay format was used in which 1 milliliter of standard lepidopteran diet was placed in each well. One hundred microliters of sample were pipetted into each well and allowed to dry. A DMSO control was included with each set of samples. Spinosad (MilliporeSigma, catalog #33706) was used as a positive control for insecticidal activity, and water was used as an additional negative control. Data were collected to construct a dose curve and determine the half-maximal lethal concentration (LC₅₀) value. Results of these experiments are provided in Table 4. In Table 4, % of insects killed is provided in the table cells for the concentrations listed at the top (2500, 625, 156, and 0 μg/mL arundine).

TABLE 4
Killing potency of arundine against insect panel

| | | % killing at | | | |
|---|---|---|---|---|---|
| | | indicated arundine | | | |
| | | concentration (μg/mL) | | | |
| Organism | Common name | 2500 | 625 | 156 | 0 |
| D. saccharalis | Sugarcane borer | 100 | 100 | 100 | 28 |
| S. eridania | Southern armyworm | 100 | 100 | 23 | 10 |
| A. gemmatalis | Velvetbean caterpillar | 97 | 70 | 100 | 10 |
| S. exigua | Beet armyworm | 72 | 94 | 72 | 13 |
| H. virescens | Tobacco budworm | 87 | 74 | 74 | 3 |
| D. grandiosella | Southwestern corn borer | 83 | 90 | 47 | 13 |
| T. ni | Cabbage looper | 93 | 72 | 41 | 10 |
| S. frugiperda | Fall armyworm | 97 | 63 | 40 | 3 |
| H. zea | Corn earworm | 90 | 50 | 40 | 9 |
| C. includens | Soybean looper | 80 | 50 | 27 | −7 |
| C. pomonella | Codling moth | 39 | 50 | 21 | −7 |

### Example 5

### Testing of BIM Compounds Against Resistant Fall Armyworm

During the development of embodiments of the technology provided herein, experiments were conducted to test the killing potency of arundine-derived BIM compounds against strains of fall armyworm (FAW) that are resistant to Bacillus thuringiensis (Bt) insecticidal proteins (e.g., Cry1Fa and Vip3A). Susceptible FAW (Spodoptera frugiperda) was obtained from Benzon Research Inc, Lancaster, PA. Cry1Fa resistant FAW originating from Puerto Rico was reared in a facility by Genective (Weldon, IL). Vip3A resistant FAW originating from southern US was reared in a facility by Genective (Weldon, IL).

FAW eggs were incubated at approximately 26° C. at 50% relative humidity until eclosion (approximately 3 days) and the hatch was timed to within 12 hours of infesting the diet plates. BIM compounds were obtained from frozen stocks, thawed, suspended in 5% DMSO along with NYSM buffer to provide a solution of 2000 ppm BIM compound in a total volume of 1.0 ml, and stored at −20° C. For testing, a single tube of each BIM compound sample was thawed at 4° C. NYSM buffer was warmed to room temperature. A volume of 750 μl. NYSM buffer was added to 750 μl from each 1.0-ml volume to prepare samples of 1.5 ml BIM compound at 1000 ppm for testing. A total of three BIM preparations per BIM molecule were prepared and tested for killing potency. Compounds tested were BIM-1, 11, 25, 26, 28, 29, 30, 32, 34. A solution of 5% DMSO in NYSM buffer was used as a negative control.

A standard commercially available semi-solid lepidopteran diet was prepared following manufacturer's instructions (Frontier, multi-species diet). Negative controls included Untreated control diet, PBS buffer, and Cry34/35 expressing Cry34 and Cry35 proteins that are inactive against FAW. LB+Kanamycin (antibiotic) growth media is the negative control for bacterial cultures. Cry1Fa is a positive control prepared as E. coli cells expressing the full-length Cry1Fa protein resuspended in PBS buffer at half the volume of the original growth culture resulting in 2× concentration of cells. Vip3A is a positive control prepared as E. coli whole cell culture expressing Vip3A.

For each of 3 replicates, 200 μl of diet was prepared in wells of a 96-well plate and the diet plates were warmed to room temperature prior to dispensing samples. After 750 μl NYSM buffer was added to 750 μl from each 1.0-ml volume to prepare samples of 1.5 ml BIM compound at 1000 ppm for testing, BIM solutions were vortexed and 20 μl were dispensed onto the diet surface in each of 16 wells using a repeating pipettor. These steps were repeated for each sample and control for each insect population being tested. BIM compound and control solutions were allowed to absorb into the diet in a biosafety hood until the diet surface was visibly dry. Once dry, wells were sealed with a clear membrane. Two pin holes were poked through the membrane in each well to maintain appropriate humidity level and aeration. Each well was infested with a single neonate larva, and the wells were resealed with perforated membrane to prevent escape. The infested plates were incubated in environmental chambers at 26° C. and 50% relative humidity for 5-7 days. All bioassays were assessed visually to record larval mortality and growth inhibition.

Two trials, spaced 1 week apart, were conducted to test killing of susceptible FAW and the two resistant varieties (Cry1 and Vip3A). In both trials, significant lethality was observed in both the susceptible FAW and the two resistant varieties. BIM-26 showed the lowest lethality of the test materials, i.e., 60-70% lethality at 7 days. BIM-11 showed 100% lethality in Vip3A-resistant neonates. BIM-28, BIM-29, and BIM-34 showed 100% lethality against Cry1 FAW. BIM-25, BIM-30, and BIM-32 showed strong lethality against both resistant species.

### Example 6

Synthesis of Asymmetric and/or Chiral Arundine Derivative Compounds

During the development of embodiments of the technology provided herein, experiments were conducted to synthesize asymmetric BIM compounds and test killing potency against FAW. In particular, BIM-36 and BIM-37 at two racemic ratios were synthesized. See Table 2 and Table 3; synthesis schemes are provided in Example 3. BIM-36 had an LD₅₀ of 2457 μM (standard deviation 811 μM); BIM-37 at an R/S ratio of 1:1 had an LD50 of 725 μM (standard deviation 138 μM); and BIM-37 at an R/S ratio of 3:1 had an LD50 of 576 μM (standard deviation 21 μM), indicating that the R enantiomer may have more killing potency than the S enantiomer.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A compound comprising a structure according to: 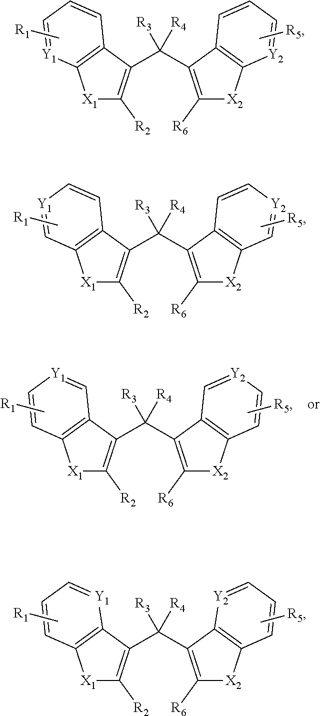
wherein:
Y₁ is N or C;
Y₂ is N or C;
R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₃ is H, alkyl, or aryl;
R₄ is H, alkyl, or aryl;
R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
X₁ is CH—R₇, N—R₇, O, or S, wherein R₇ is H, alkyl, or aryl; and
X₂ is CH—R₈, N—R₈, O, or S, wherein R₈ is H, alkyl, or aryl.

2. The compound of claim 1, wherein R₃ or R₄ comprises an aryl.

3. The compound of claim 1, wherein R₃ or R₄ comprises an aryloxy.

4. The compound of claim 1, wherein R₃ or R₄ comprises a phenoxy.

5. The compound of claim 1, wherein R₁ or R₅ comprises a halogen.

6. The compound of claim 1, wherein R₁ or R₅ comprises a fluorine.

7. The compound of claim 1, wherein X₁ or X₂ is N.

8. The compound of claim 1, wherein Y₁ or Y₂ is C.

9-23. (canceled)

24. A method of synthesizing an insecticidal compound, the method comprising:
reacting a heterocyclic molecule and a ketone or aldehyde.

25. The method of claim 24, wherein said heterocyclic molecule comprises an indole.

26. The method of claim 24, wherein said insecticidal compound is an arundine analog.

27. The method of claim 24, wherein said insecticidal compound is a bisindole compound.

28. The method of claim 24, wherein said insecticidal compound comprises a structure according to: 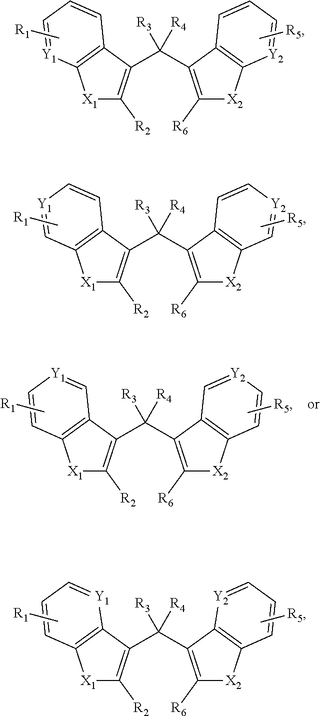
wherein:
Y₁ is N or C;
Y₂ is N or C;
R₁ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₂ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₃ is H, alkyl, or aryl;
R₄ is H, alkyl, or aryl;
R₅ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
R₆ is H, alkyl, aryl, O-alkyl, O-aryl, O-acyl, N-alkyl, N-aryl, N-acyl, S-alkyl, S-aryl, S-acyl, I, Br, Cl, F, CN, CF₃, or NO₂;
X₁ is CH—R₇, N—R₇, O, or S, wherein R₇ is H, alkyl, or aryl; and
X₂ is CH—R₈, N—R₈, O, or S, wherein R₈ is H, alkyl, or aryl.

29.-30. (canceled)

31. A method of killing an insect, said method comprising contacting an insect with a compound of claim 1.

32. The method of claim 31, wherein the compound is provided at a concentration of less than 30 micromolar.

33. The method of claim 31, wherein the compound is provided at a concentration of less than 20 micromolar.

34. The method of claim 31, wherein the compound is provided at a concentration of less than 10 micromolar.

35. The method of claim 31, wherein contacting an insect comprises ingesting by said insect, contacting a cuticle or exoskeleton of said insect, or passing through a spiracle of said insect.

36. An insecticidal composition comprising a compound of claim 1.

37. The insecticidal composition of claim 36, wherein the compound is present at a weight percentage of from 0.1% to 99.9%.

38-43. (canceled)
